(19) United States
(12) Patent Application Publication
(10) Pub. No.: US 2026/0308699 A1
(43) Pub. Date: Oct. 8, 2026

(54) METHODS AND SYSTEMS FOR IDENTIFICATION OF SEQUENCE ADJACENT MOTIF, POTENCY, AND FIDELITY OF A NUCLEASE

(71) Applicant: GENEDITBIO LIMITED, Hong Kong (CN)

(72) Inventors: Zongli Zheng, Hong Kong (CN); Miao Yu, Hong Kong (CN)

(21) Appl. No.: 19/482,814

(22) PCT Filed: May 9, 2024
(86) PCT No.: PCT/CN2024/092025

§ 371 Date: Nov. 10, 2025

Related U.S. Application Data
(60) Provisional application No. 63/500,916, filed on May 9, 2023.

(51) Int. Cl. C12Q 1/689; C12N 9/22; C12N 15/11

(52) U.S. Cl. CPC C12Q 1/689; C12N 9/226; C12N 15/11; C12N 2310/20; C12Q 2600/156

(57) ABSTRACT

Provided is a method for identification of sequence adjacent motif, potency, and fidelity of a nuclease.

### CROSS-REFERENCE TO RELATED APPLICATIONS

This application claims the priority to provisional patent application U.S. 63/500,916. The entire contents of the aforementioned applications are hereby incorporated by reference.

### FIELD OF INVENTION

This invention relates to methods for identification of sequence adjacent motif, potency, and fidelity of a nuclease.

### BACKGROUND OF INVENTION

Nuclease is an enzyme capable of cleaving the phosphodiester bonds between nucleotides of nucleic acids. Clustered regularly interspaced short palindromic repeats (CRISPR)-associated (Cas) systems are found in a wide range of bacteria and archaea and typically consist of a set of CRISPR arrays and CRISPR associated genes. CRISPR arrays contain direct repeats and spacers that are transcribed and processed to form CRISPR RNA (crRNA), which guides Cas proteins to specific locations in the genome. In prokaryotes, CRISPR-Cas proteins provide antiviral immunity through DNA sequence recognition and disruption of intruding viral DNA.

The CRISPR-Cas system recognizes sequence target at the presence of a protospacer adjacent motif (PAM). To identify PAM sequence specificity of a CRISPR-Cas nuclease, various in vivo and in vitro methods have been used previously, such as in vitro cleavage assay, HT-PAMDA, PAM-SCANR and GFP-based assays. Each of them is associated with different limitations. Certain attempts have been made in providing a positive selection assay. However, this method requires a tedious prior purification of the candidate protein that may or may not work; the cleavage is purely biochemical and may not recapitulate the situations in a living organism, such as in bacteria or in a mammalian cell. Other attempts have been made in providing an in vitro method named HT-PAMDA. However, like some other negative selection methods, HT-PAMDA is more appropriate for identifying Cas proteins with defined PAM sequence, instead of those with nearly random PAM sequences. Moreover, contacting the DNA library with cell lysis is prone to degradation of the DNA fragments added. Other attempts have been made in providing in vivo methods like PAM-SCANR and GFP assay. However, PAM-SCANR is based on dead Cas nuclease, which is different from cleavage-based analysis. Fluorescence (GFP or RFP)-based methods use the chance restoration of protein expression frame after nuclease-induced insertion or deletion, followed by cell sorting and next-generation sequencing to identify candidate PAM sequences. However, they are associated with particularly low efficiency, as previous GFP assays have shown that often far less than 1% of experiment cells might contain mixed candidate PAM sequences for downstream analyses. Maintaining high-diversity sequence libraries in cell culture is challenging.

### SUMMARY OF INVENTION

This disclosure provides a method for identification of adjacent sequence motif of a nuclease using a sequence that occurs more than once in a genome, wherein the method comprises the steps of:
(1) identifying a repeat sequence from a genome of interest, where the flanking sequences to the repeat sequence are diversified;
(2) contacting a nuclease of interest with the genome to target the repeat sequence; and
(3) analyzing the flanking sequences of modified or unmodified repeat sequence(s), so as to identify the adjacent sequence motif of the nuclease.

This disclosure also provides a method for identification of editing efficiency of a nuclease using a sequence that occurs more than once in a genome, wherein the method comprises the steps of:
(1) identifying a repeat sequence from a genome of interest, where the flanking sequences to the repeat sequence are diversified;
(2) contacting a nuclease of interest with the genome to target the repeat sequence; and
(3) measuring the editing efficiency of the target loci.

This invention also provides a method for identification of off-target of a nuclease using a sequence that occurs more than once in a genome, wherein the method comprises the steps of:
(1) identifying a repeat sequence from a genome of interest, where the flanking sequences to the repeat sequence are diversified;
(2) contacting a nuclease of interest with the genome to target the repeat sequence; and
(3) quantifying the edited sequences with perfectly matched (on-targets) and with mismatched bases (off-targets).

In some embodiments, the nuclease comprises a clustered regularly interspaced short palindromic repeats-associated protein (Cas protein) and the adjacent motif is a protospacer adjacent motif (PAM). In some embodiments, the nuclease in step (2) comprises a CRISPR-Cas protein (Cas protein), a guide-RNA containing the repeat sequence or repeat's complementary sequence is used to direct the Cas protein to target the repeat.

In some embodiments, the nuclease is in the form of DNA plasmid and is directly expressed in a living cell. In some embodiments, the nuclease is in the form of mRNA and is translated in a living cell. In some embodiments, the genome of interest is a eukaryotic genome; optionally, the eukaryotic genome is from human, mouse, rat, pig, monkey, or other animals, plants, fungi, or protists. In some embodiments, the genome of interest is a prokaryotic genome; optionally, the prokaryotic genome is from bacterial or archaea.

In some embodiments, the Cas protein is a Type II protein; optionally, the Type II protein is a Cas9, or its variants; preferably the Cas9 protein is Streptococcus pyogenes Cas9 (SpCas9), Staphylococcus aureus (SaCas9), or their variants. In some embodiments, the Cas protein is a Type V protein; preferably, the Type V protein is selected any one of Cas12a, Cas12b, Cas12c, Cas12d, Cas12e, Cas12f (also known as Cas14), Cas12g, Cas12h, Cas12i, Cas12j, Cas12k, or their variants. In some embodiments, the Cas protein is a Type VI protein; preferably, the Type VI protein is CasRx, C2c2, or their variants. In some embodiments, the length of the PAM is 1 to 10 bases, 11 to 20 bases, or 21 to 100 bases. In some embodiments, the length of the PAM is 1, 2, 3, 4, 5, 6, 7, 8, 9, or 10 bases. In some embodiments, the PAM identified is used for CRISPR-based gene-editing therapeutic applications. In some embodiments, the PAM identified is used for nuclease-based molecular diagnosis applications.

In some embodiments, the method further comprises the step of: quantifying the edited sequences with perfectly matched (on-targets) and with mismatched bases (off-targets) with the repeat sequence to infer, under given experiment conditions, the potency of the nuclease that is correlated with the number of edited loci in a genome and the total number of edited DNA fragments at the on-target and off-target loci.

In some embodiments, the method further comprises the step of: quantifying the edited sequences with perfectly matched (on-targets) and with mismatched bases (off-targets) with the repeat sequence to infer, under given experiment conditions, the fidelity of the nuclease that is correlated with the proportion of edited on-target loci in the genome and the total number of edited DNA fragments at these on-target loci relative to the off-target loci and off-target DNA fragments, respectively.

This disclosure also provides a polynucleotide for a specific application, wherein the polynucleotide comprises a spacer sequence a nucleic acid sequence encoding such spacer sequence, wherein the spacer sequence is capable of binding to a repeat sequence from a genome of interest and the flanking sequences to the repeat are diversified; wherein the specific application is identification of adjacent sequence motif of a nuclease, identification of editing efficiency of a nuclease, or identification of off-target of a nuclease.

In some embodiments, the polynucleotide is a gRNA comprising a spacer sequence or a nucleic acid sequence encoding such gRNA. In some embodiments, the gRNA further comprises a sequence capable of forming complex with the nuclease. In some embodiments, the gRNA further comprises a scaffold sequence. In some embodiments, the polynucleotide is a gRNA comprising a spacer sequence and a scaffold sequence. In some embodiments, the polynucleotide comprises a nucleic acid sequence encoding a gRNA comprising a spacer sequence and a scaffold sequence. In some embodiments, the gRNA is a dual-guide RNA (dgRNA). In some embodiments, the gRNA is a single-guide RNA (sgRNA). In some embodiments, the gRNA is a modified gRNA. In some embodiments, only one gRNA as disclosed in the present invention is configured to facilitate the identification or selection of protospacer adjacent motif (PAM) sequences recognized by variants of a Cas protein, such as those derived from SpCas9, thereby enabling efficient screen across a spectrum of Cas protein variants with diverse PAM compatibilities. In typical embodiments, the gRNA is capable of forming complex with the Cas protein. In some embodiments, the polynucleotide comprises a nucleic acid sequence encoding the gRNA thereof.

In some embodiments, the spacer sequence is complementary to at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 consecutive bases of the repeat sequence or the repeat's complementary sequence within the genome. In some embodiments, the repeat sequence has an occurrence of more than 4, 16, 64, 256, 1024, 4096, or 16384 times in the genome. In some embodiments, the repeat sequence has an occurrence of more than 4, 10, 16, 20, 30, 40, 50, 60, 70, 80, 90. 100, 110, 120, 130, 140, 150, 160, 170, 180,190, 200, 250, 300, 350, 400, 450, 500, 500, 600, 700, 800, 900, 100, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 100000, 110000, 120000, 130000, 140000, 150000, 160000, 170000, 180000, 190000, 200000, 210000, 220000, 300000, 310000, 320000, 330000, 340000, 350000, 360000, 370000, 380000, 390000, 400000, 410000, 420000, 430000, 440000, 450000, 460000, 470000, 480000, 490000, 500000 times in the genome. In some embodiments, the repeat sequence comprises the any one of the sequences as set forth in SEQ ID NO: 1, 8-480. Further attributes of the repeat sequence disclosed in this invention are also encompassed within this section.

This disclosure also provides a CRISPR-Cas system comprising the polynucleotide herein. In some embodiments, the CRISPR-Cas system further comprises a Cas protein or a nucleic acid encoding the Cas protein. In some embodiments, the Cas Protein is selected from a Type II protein; optionally, the Type II protein is a Cas9, or its variants; preferably the Cas9 protein is Streptococcus pyogenes Cas9 (SpCas9), Staphylococcus aureus (SaCas9), or their variants. In some embodiments, the Cas protein is a Type V protein; preferably, the Type V protein is selected any one of Cas12a, Cas12b, Cas12c, Cas12d, Cas12e, Cas12f (also known as Cas14), Cas12g, Cas12h, Cas12i, Cas12j, Cas12k, or their variants. In some embodiments, the Cas protein is a Type VI protein; preferably, the Type VI protein is CasRx, C2c2, or their variants. Further attributes of the Cas protein disclosed in this invention are also encompassed within this section.

This disclosure also provides a vector comprising a nucleic acid encoding the polynucleotide herein. In some embodiments, the vector is a plasmid.

This disclosure also provides a vector system comprising a nucleic acid encoding the polynucleotide herein and a nucleic acid sequence encoding a Cas protein herein. In some embodiments, the nucleic acid encoding the polynucleotide and the nucleic acid sequence encoding the Cas protein are placed in a same vector. In some embodiments, the nucleic acid encoding the polynucleotide and the nucleic acid sequence encoding the Cas protein are placed in different vectors. In some embodiments, the vector is a plasmid.

### BRIEF DESCRIPTION OF FIGURES

FIG. 1 (FIG. 1). Method design. (a) SeqLogo plot showing nucleotide frequency at each position in the 8 bases upstream (positions 1 to 8) and downstream (positions 29-36) flanking the constant Rep-1R sequence. Rep-1R and Rep-1 sequences can be used to characterize PAM preferences of Cas nucleases with 5′ PAM and 3′ PAM, respectively. (b) PAM assay workflow for the identification of PAM preferences using the highly repetitive genome sequence Rep-1 as the protospacer and the GUIDE-Seq experiments to capture cleaved genomic sites of SpCas9. (c) An example of GUIDE-seq output. Each line showed one SpCas9 cleavage site and the mismatched bases were color coded. The flanking sequences and GUIDE-seq read counts of each site were shown on the right side. (d) SeqLogo plot summary for SpCas9 PAM preferences, using their corresponding read counts as weights and stratified by perfect match and mismatch targets.

FIG. 2 (FIG. 2). Identified PAM sequences. (a, c, e, g, h) Sequence-logo results for SpCas9, SaCas9, FnCas12a, CjCas9 and SpRY Cas proteins in 293T cells. The left part was summarized by perfect matched (PM) model and the right part was summarized by mismatched (MM) model. (b, d, f) Sequence logo results for SpCas9, SaCas9, FnCas12a in HepG2 cells. The left part was summarized by perfect matched (PM) model and the right part was summarized by mismatched (MM) model.

FIG. 3 (FIG. 3). Novel Cas nuclease discovery and its PAM identification using the Genome-PAM assay. (a) A schematic illustration of the genomic DNA region from the Type V-A CRISPR-Cas system. (b) SeqLogo results for RuCas12a PAM preferences in HEK293T cells with perfect match spacers (left) and mismatch spacers (right). (c) Editing efficiency of RuCas12a on 20 regular genomic sites in human genes CD34, CFTR, DNMT1, EMX1, HBB, LPA, POLQ, RFN2, TTR and VEGFA with a 5′-TTTG PAM.

FIG. 4 (FIG. 4). General activities and specificities of different SpCas9 variants. (a) The number of on-target sites to the number of off-target sites of seven SpCas9 variants. (b) The number of on-target sequencing reads to the number of off-target sequencing reads of seven SpCas9 variants. The number of on-target sites (c) and the number of off-target sites (d) detected when using randomly down-sampled datasets, from 0.1 M to 1 M raw sequencing reads. (e) Relative activities (defined as the number of perfect match sites relative to that of the WT SpCas9) and specificities (defined as the ratio of perfect match to mismatch site numbers relative to the ratio in SpCas9-HF1) of seven SpCas9 variants.

FIG. 5 (FIG. 5). The Genome-PAM assay profiles of chromosome accessibility when using SpCas9 and targeting ‘Rep-1R’ in human cell lines Hela, HepG2, and Huh7. The proportion of cleavage read counts in each five-million base chromosomal window was divided by the proportion in the corresponding chromosomal window in the HEK293T cells (mean of triplicates) and log 2-transformed. A higher cleavage proportion relative to HEK293T was colored in red and a relatively lower proportion in blue.

### DETAILED DESCRIPTION

The following examples further illustrate the present disclosure, but the present disclosure is not limited thereto.

### 1. General Definitions

Unless defined otherwise, the technical and scientific terms used herein have the same meaning as commonly understood by one of ordinary skill in the art to which this disclosure pertains.

When “t” or “T” appears in a sequence in this invention as a nucleotide of an RNA sequence, it should be understood as “u” or “U”.

As used herein, the term “a”, “an”, “the”, and “said” and similar terms used in the context of the present disclosure (especially in the context of the claims) are to be construed to cover both the singular and plural unless otherwise indicated herein or clearly contradicted by the context. In addition, it should be noted that the plural form does not necessarily mean that it is plural, and it needs to be understood according to the context in the article.

The use of “or” or “/” is inclusive and means “and/or” unless stated otherwise; or it could be interpreted differently based on the context.

The term “identity” in the context of two or more nucleic acids or polypeptide sequences refers to two or more sequences or subsequences that are the same or have a specified percentage of amino acid residues or nucleotides that are the same as measured using a BLAST or BLAST 2.0 or FASTA etc. sequence comparison algorithms with default parameters described below.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and those terms such as “consisting essentially of” and “consists essentially of” have the meaning ascribed to them in U.S. Patent law.

The term “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about”, as used herein when referring to a measurable value such as a parameter, an amount, a temporal duration, and the like, are meant to encompass variations of and from the specified value, insofar such variations are appropriate to perform in the disclosed disclosure. It is to be understood that the value to which the modifier “about” refers is itself also specifically, and preferably, disclosed.

The term “exemplary” is used herein to mean serving as an example, instance, or illustration. Any aspect or design described herein as “exemplary” is not necessarily to be construed as preferred or advantageous over other aspects, embodiments, or designs.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “in some embodiment(s)”, “in certain embodiment(s)”, “in some preferred embodiments”, “in some typical embodiment(s)”, “in typical embodiment(s)” or similar expressions means that a particular feature, structure or characteristic described in connection with the embodiment is included in at least one embodiment of the present disclosure. Furthermore, a particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any one of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### 2. The Method

This disclosure provides a method for identification of adjacent sequence motif (adjacent motif) of a nuclease using a sequence that occurs more than once in a genome, and the method comprising the steps of:
(1) identifying a repeat sequence from a genome of interest, where the flanking sequences to the repeat sequences are diversified;
(2) contacting a nuclease of interest with the genome to target the repeat sequence; and
(3) analyzing the flanking sequences of modified or unmodified repeat sequence(s), so as to identify the adjacent sequence motif of the nuclease.

This disclosure also provides a method for identification of editing efficiency of a nuclease using a sequence that occurs more than once in a genome, wherein the method comprises the steps of:
(1) identifying a repeat sequence from a genome of interest, where the flanking sequences to the repeat sequence are diversified;
(2) contacting a nuclease of interest with the genome to target the repeat sequence; and
(3) measuring the editing efficiency of the target loci.

This invention also provides a method for identification of off-target of a nuclease using a sequence that occurs more than once in a genome, wherein the method comprises the steps of:
(1) identifying a repeat sequence from a genome of interest, where the flanking sequences to the repeat sequence are diversified;
(2) contacting a nuclease of interest with the genome to target the repeat sequence; and
(3) quantifying the edited sequences with perfectly matched (on-targets) and with mismatched bases (off-targets).

Several methods are available for identifying repeat sequences in a genome of interest. Some commonly used techniques are known to those skilled in the art of bioinformatics and molecular genetics which include: Repeat Masker, BLAST, REPuter, Tandem Repeats Finder (TRF), PILER-DF, RepeatModeler, Graph-based methods, k-mer based methods, Bioinformatic Tools for Long-Read Sequencing Data, Molecular Cloning and Sequencing, Southern Blotting, FISH (Fluorescence In Situ Hybridization), Microarray Analysis, or Next-Generation Sequencing (NGS) Technologies. In some embodiments, jellyfish tools are used to calculate the frequencies of the sequences. In some embodiments, BWA are used to map their chromosomal coordinates; In some embodiments, samtools are used to retrieve the upstream and downstream. In some embodiments, the diversity of the flanking sequences of each of sequence is plotted using the ggseqlogo package. In some embodiment, the methods thereof can be used individually or in combination to achieve a comprehensive identification of repeat sequences in a genome. The choice of identifying method often depends on the type of genome, the scale of the analysis, and the specific goals of the study.

To identify the repeat sequence, in certain embodiments, the provided method uses a length or lengths of the repeat that are similar as or longer than the length of the recognition or target sequence of the candidate nuclease. In some embodiments, for CRISPR-Cas nucleases, the length of the repeat sequence is about 15 to 26 bases, and preferably between 17 to 24 bases.

In some embodiments, the adjacent motif is a protospacer adjacent motif (PAM). The genomic repeats in the genome of interest are utilized in the method herein to direct PAM identification. Genomic repeats with a diverse flanking sequence representing the candidate PAM sequences are provided. When the flanking sequences are described as “diversified,” it indicates that there is significant variation in the sequence compared to the more conserved central repeat sequence. This diversity could be a result of natural selection, genetic drift, or other evolutionary forces, as well as due to biological processes such as recombination or gene conversion. The PAM candidates can be at the 5′ or 3′ of the repeats for identification of CRISPR-Cas nucleases with 5′ PAM or 3′ PAM, respectively. In some certain embodiments, these repeat sequences are used as the protospacers to guide CRISPR-Cas nucleases. In some embodiments, to identify the PAM, a regular CRISPR-Cas editing is performed with the genomic repeats as the guides and followed by analysis of the edited sequences in the genome such as the Genome-wide, Unbiased Identification of DSBs Enabled by Sequencing (GUIDE-seq), Anchored Multiplex PCR (AMP-seq) or other methods. The methods of this disclosure provide a high-throughput screening method for PAMs having practical applications. In some embodiments, the PAM identified is used for CRISPR-based gene-editing therapeutic applications. In some embodiments, the PAM identified is used for nuclease-based molecular diagnosis applications.

Any methods in the art leading to contact a nuclease of interest with the genome are included in this disclosure. In preferred embodiments, such “contacting” or “contact” is realized through transfection. Typically, the cells are combined with an exogenous nucleic acid sequence encoding or translating the proteins herein and treated in order to accomplish transfection. As used herein, the term “transfection” includes a variety of techniques for introducing an exogenous nucleic acid into a cell including calcium phosphate or calcium chloride precipitation, microinjection, gene gun, DEAE-dextrin-mediated transfection, lipofection (e.g. Lipofectamine-mediated transfection), electroporation, LNP-mediated transfection, or virus-mediate transfection (e.g., lentiviral vectors).

In some typical embodiments, the nuclease is a CRISPR-Cas protein, a guide-RNA (gRNA) comprising a sequence capable of binding to the repeat sequence or repeat's complementary sequence, or the nucleic acid encoding such guide-RNA, is used to the direct the Cas protein to target the repeat. In some embodiments, a nucleic acid encoding the nuclease is used to realize the contacting target sequence, in such embodiments, the nucleic acid will encode the corresponding protein after transfection. In some embodiments, the spacer sequence is complementary to at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 consecutive bases of the repeat sequence or repeat's complementary sequence within the genome. In some embodiments, the spacer sequence of the gRNA comprises the repeat sequence or repeat's complementary sequence of the genome. In some embodiments, a guide-RNA containing the repeat sequence or repeat's complementary sequence, or a nucleic acid encoding such guide-RNA, is utilized to direct the Cas protein to target the repeat. In some embodiment, contacting the CRISPR-Cas protein or nucleic acid sequence encoding such CRISPR-Cas protein through transfection. In some embodiment, the gRNA or the nucleic acid sequence encoding such gRNA is transfected to direct the Cas protein to target the repeat sequence.

In some embodiments, the method in this disclosure facilitates high-throughput PAM specificity identification of many Cas nucleases as it does not require protein purification, and the Cas nucleases are expressed in genome of interest.

In some embodiments, a nucleic acid encoding the nuclease is utilized to facilitate such contacting with the genome. In some embodiments, the nuclease is in the form of DNA plasmid. In some embodiments, the nuclease is in the form of mRNA. When refer to the term “the nuclease is in the form of” a DNA plasmid or mRNA, it shall denote that the method involves utilizing DNA plasmids or mRNA encoding the nuclease of interest to perform the step of contacting said nuclease with the genome.

The method in this disclosure is highly efficient and accurate because every cell contains one, and just one, full set of candidate PAM library. In some typical embodiments, the method is performed in the living cells. Compared with in vitro methods, the method described here can eliminate the need of protein purification, avoiding the fast degradation of DNA templates in cell lysis, and is not subject to the known differences in sequence preferences between biochemical versus in cell cleavages, between prokaryotic and mammalian cells. In some embodiments, the nuclease is in the form of DNA plasmid and is directly expressed in a living cell. In some embodiments, the nuclease is in the form of mRNA and is translated in a living cell. In some embodiments, the living cell is a eukaryotic cell. In some embodiments, the eukaryotic cell is selected from the group consisting of cells from human, mouse, rat, pig, monkey, or other animals, plants, fungi, or protists. In some embodiments, exemplary human cell lines include 3T3; A375; A431; A549; Daudi; HEK293; Huh7; HeLa; HepaRG; HepG2; Jurkat; MDA-MB-231; MDA-MB-436; MDA-MB-468; Saos-2; 1321 N1; AtT-20; B16; Ba/F3; BHK; Caki; Calu; CHO; COS; CV-1; Detroit; DMS; EPH4; HEK293T; HL-60; HUVEC; K562; Kasumi; LLC-MK2; MCF; MDA-MB; MDCK; PC3 (PC-3); Phoenix; SCC; Sf21; Sf9; SNU; T47D; THP1; U937 (U-937); U2-OS; and Vero cells. Methods for expressing or translating protein in cells are well known in the art.

Any method in the art for analyzing flanking sequences of the modified or unmodified sequence(s) or measuring the editing efficiency of the target loci is included in this disclosure. In some embodiments, the method for measuring the editing efficiency includes sequencing analysis (e.g., Sanger Sequencing, Next-Generation Sequencing (NGS)), PCR and agarose gel electrophoresis, mismatch cleavage assays, high-resolution melting (HRM) analysis, Fluorescence In Situ Hybridization (FISH) analysis, immunostaining and flow cytometry, single-cell sequencing, digital PCR (dPCR), or functional assays. In some embodiments, method for during the editing efficiency may comprise at least one step of the following steps of: sampling and DNA extraction, amplification of target loci, sequencing of edited loci, analysis of sequencing data, quantification of on-Target and off-Target events, validation by Sanger sequencing, phenotypic analysis, statistical analysis; or reporting. In some embodiments, the Genome-wide, Unbiased Identification of DSBs Enabled by Sequencing (GUIDE-seq), Anchored Multiplex PCR (AMP-seq) or other methods are used to analyze the flanking sequences. In some embodiment, the methods thereof can be used individually or in combination. Furthermore, by analyzing the number of loci and sequencing reads, the provided method also allows for simultaneously assessing the potency (on-target efficiency) and fidelity (off-target effects) of the nuclease.

Any method for quantifying the edited sequences with perfectly matched (on-targets) and with mismatched bases (off-targets) is included in this disclosure. In some embodiments, the method for quantifying the edited sequences with perfectly matched (on-targets) and with mismatched bases (off-targets) includes: dual luciferase reporter assay; Next-Generation Sequencing (NGS) technologies, bioinformatics analysis, digital PCR (dPCR), restriction enzyme digestion, single-cell sequencing, immunostaining and flow cytometry, High-Resolution Melting (HRM) analysis, induced Pluripotent Stem Cell (iPSC) Differentiation, or functional assays. In some embodiments, the Genome-wide, Unbiased Identification of DSBs Enabled by Sequencing (GUIDE-seq), Anchored Multiplex PCR (AMP-seq) or other methods are used. In some embodiment, the methods thereof can be used individually or in combination.

In some embodiments, method of analyzing flanking sequences of the modified or unmodified sequence(s), measuring the editing efficiency or quantifying the edited sequences with perfectly matched (on-targets) and with mismatched bases (off-targets) includes: the GUIDE-Seq (Genome-wide, Unbiased Identification of DSBs Enabled by Sequencing), DISCOVER-Seq (Discovery of in situ Cas off-targets and verification by sequencing), or EDITED-Seq (editing events detection by sequencing). The entire content of WO2022243748A2 is incorporated herein by reference.

In some embodiments, the method further comprises the step of quantifying the edited sequences with perfectly matched (on-targets) and with mismatched bases (off-targets) with the repeat sequence to infer, under given experiment conditions, the potency of the nuclease that is correlated with the number of edited loci in a genome and the total number of edited DNA fragments at the on-target and off-target loci. In some embodiments, the method further comprises the step of: quantifying the edited sequences with perfectly matched (on-targets) and with mismatched bases (off-targets) with the repeat sequence to infer, under given experiment conditions, the fidelity of the nuclease that is correlated with the proportion of edited on-target loci in the genome and the total number of edited DNA fragments at these on-target loci relative to the off-target loci and off-target DNA fragments, respectively.

In some embodiments, the term “under given experiment conditions” shall refer to a set of predetermined parameters and variables that are maintained consistently throughout the experimental procedure. These may include specific nuclease types and versions, cell lines originating from human, porcine, rabbit, or other sources, detailed protocols for conducting the experiments, environmental factors such as temperature, pH, and salt concentration, duration and stages of the experiment, and the application of particular methodologies like GUIDE-seq, AMP-seq, or other techniques used for the detection and quantification of edited sequences. The definition of ‘given experiment conditions’ ensures that the inferred potency or fidelity of the nuclease is applicable only to the specific conditions under which the experiments are conducted. In some embodiments, “under given experiment conditions” shall be understand as it is commonly used in English expression.

### 3. Nuclease

The methods include contacting a nuclease of interest with the genome. In some typical embodiments, the genome is modified by the nuclease with a guide RNA (gRNA) comprising a spacer sequence. The term “nuclease” herein refers to any enzyme that can modify a nucleic acid, including variants of such enzymes and genome editing reagents based on such enzymes, such as Cas9 proteins, base editors, or prime editors. A number of such reagents, and methods for creating variants, are known in the art.

In some preferred embodiments, the nuclease is a CRISPR-Cas protein (Cas protein or Cas nuclease). In some embodiment, the Cas protein is or comprises a Type II protein, such as Streptococcus pyogenes Cas9 (SpCas9) or Staphylococcus aureus (SaCas9), or their variants. In some embodiments, the Cas protein is or comprises a Type V protein, such as a Cas12a, Cas12b, Cas12c, Cas12d, Cas12e, Cas12f (also known as Cas14), Cas12g, Cas12h, Cas12i, Cas12j, Cas12k, or their variants. In some embodiments, the Cas protein is or comprises a Type VI protein, such a CasRx, C2c2, or their variants. In some embodiments, the Cas protein herein includes other Cas proteins not explicitly listed in this application but known to those skilled in the art, including their variants.

“Variant” of a protein or a polynucleotide disclosed herein means sequence of such protein or polynucleotide having one or more additions, deletions, stop positions, or substitutions, as compared to the original sequence. An amino acid substitution can be a conservative or a non-conservative substitution. Variant of protein or peptide sequences disclosed herein can include those having one or more conservative amino acid substitutions. One of skill in the art could identify and generate such variants.

In some embodiments, the nuclease possesses both DNA/RNA binding activity and nucleic acid cleavage activity. In some embodiments, the nuclease exhibits DNA/RNA binding activity in conjunction with a nicking activity, without necessarily cleaving the nucleic acid completely (e.g. a Cas9 nickase). In some embodiments, the nuclease is characterized solely by DNA/RNA binding activity, devoid of any cleavage activity. In some embodiments, a functional domain, which is capable of modifying a DNA or RNA molecule, is linked to such nuclease to modify the genome. The functional domain can have one or more types of enzymatic activities, including polymerase activity, ligase activity, reverse transcriptase activity, deaminase activity, replication activity, or proofreading activity; In some embodiment the functional domain comprises a nuclease, a nickase, a deaminase, a reverse transcriptase, a recombinase, a methyltransferase, a methylase, an acetylase, an acetyltransferase, a transcriptional activator, or a transcriptional repressor domain.

Any way in the art leading to contact a nuclease of interest with the genome is included in this disclosure. In some embodiments, a nucleic acid encoding the nuclease is utilized to facilitate such contacting with the genome. In some embodiments, the nuclease is in the form of DNA plasmid. In some embodiments, the nuclease is in the form of mRNA. In some embodiments, contacting the nuclease of interest with the genome is performed in a living cell. In some embodiments, the nuclease is in the form of DNA plasmid and is directly expressed in a living cell. In some embodiment, the nuclease is in the form of mRNA and is translated in a living cell. When refer to the term “the nuclease is in the form of” DNA plasmid or mRNA, it shall denote that the method involves utilizing DNA plasmids or mRNA encoding the nuclease of interest to perform the step of contacting said nuclease with the genome.

In some typical embodiments, the nuclease is a CRISPR-Cas protein, a guide-RNA (gRNA) comprising a sequence capable of binding to the repeat sequence or repeat's complementary sequence, or the nucleic acid encoding such guide-RNA, is used to the direct the Cas protein to target the repeat. In some embodiments, the spacer sequence is complementary to at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 consecutive bases of the repeat sequence or repeat's complementary sequence within the genome. In some embodiments, the spacer sequence of the gRNA comprises the repeat sequence or repeat's complementary sequence of the genome. In some embodiments, a guide-RNA containing the repeat sequence or repeat's complementary sequence, or a nucleic acid encoding such guide-RNA, is utilized to direct the Cas protein to target the repeat.

Any methods in the art leading to direct the Cas protein to target the repeat is included in this disclosure. In some embodiments, a nucleic acid encoding the guide-RNA is utilized to facilitate directing the Cas protein to target the repeat. In some embodiments, the guide-RNA is in the form of DNA plasmid. In some embodiments, directing the Cas protein is performed in a living cell. In some embodiments, the guide-RNA is in the form of DNA plasmid and is directly expressed in a living cell. In some embodiments, the gRNA is chemically synthesized. In some embodiments, the gRNA is a modified gRNA.

### 4. Repeat Sequences in the Genome

The “genome”, as used herein, refers to the complete set of genes or genetic material present in a cell, tissue or organism. In some embodiments, the genome comprises a DNA. In some embodiments, the DNA is a double-strand DNA. In some embodiments, the DNA is a single-strand DNA. In some embodiment, the DNA is a cDNA. In some embodiments, the genome comprises an RNA (e.g., an mRNA). In some embodiments, the genome of interest is a eukaryotic genome, for example human, mouse, rat, pig, monkey, or other animals, plants, fungi, or protists.4

Repeat sequences (repeats) in the genome refers to sequences that occur more than once in a genome. The repeat sequence can be short or long. In some embodiments, the repeat sequence is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, for CRISPR-Cas nucleases, the length of the repeat sequence is about 15 to 26 bases, and preferably between 17 to 24 bases.

The repeat sequence may be exact copies (perfect repeats) or similar but not identical (imperfect repeats). In preferred embodiments, the repeat sequence is a perfect repeat. The repeat sequence can occur naturally or can be introduced artificially, such as through genetic editing techniques. In preferred embodiments, the repeat sequence is naturally occurred.

Various repeats in a genome of interest might be used for sequence motif identification. One consideration is the potential length of the motif, typically ranges from 1 to 8 nucleotides as for the PAM sequence in CRISPR-Cas systems. In some embodiments, the length of the PAM is 1 to 10 bases, 11 to 20 bases, or 21 to 100 bases. In some embodiments, the length of the PAM is 1, 2, 3, 4, 5, 6, 7, 8, 9, or 10 bases. In some embodiments, the diversity of the sequence library, which is related with the number of unique flanking sequences to a genomic repeat sequence, comprises potential sequence motifs to be tested. For example, the PAM of Streptococcus pyogenes Cas9 (SpCas9) and its variants may range from 1 to 3 bases, therefore the number of unique flanking sequences in one genome would preferably be 64 (=4\hat{ }3) or more, and with even proportions of different sequences. In the case of Staphylococcus aureus (SaCas9) and its variants, the PAM length may range from 5 to 6 bases, therefore the number of unique flanking sequences would preferably be 4\hat{ }5 (=1024) to 4\hat{ }6 (=4096) or more, and with even proportions of different sequences.

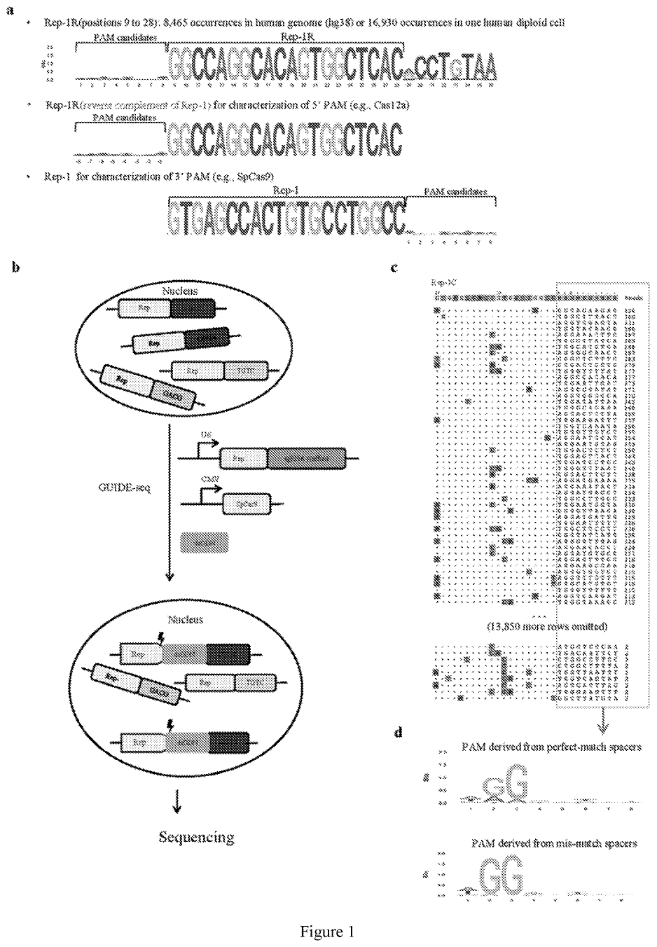

In some embodiments, the repeat has an occurrence of more than 4, 16, 64, 256, 1024, 4096, 16384, 65536, 262144, or 500000 times in the genome. In some embodiments, the repeat sequence has an occurrence of more than 4, 10, 16, 20, 30, 40, 50, 60, 70, 80, 90, 100, 110, 120, 130, 140, 150, 160, 170, 180,190, 200, 250, 300, 350, 400, 450, 500, 500, 600, 700, 800, 900, 100, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10000, 20000, 30000, 40000, 50000, 60000, 70000, 80000, 90000, 100000, 110000, 120000, 130000, 140000, 150000, 160000, 170000, 180000, 190000, 200000, 210000, 220000, 300000, 310000, 320000, 330000, 340000, 350000, 360000, 370000, 380000, 390000, 400000, 410000, 420000, 430000, 440000, 450000, 460000, 470000, 480000, 490000, 500000 times in the genome (any integer between the above-mentioned numbers should be considered as being included/documented in this disclosure). Table 1 lists some example repeats, which has an occurrence of 8,465 times in human genome (version hg38) and the flanking sequences are evenly distributed as indicated by the sequence logo plot (FIG. 1) showing no dominant nucleotides flanking the 5′ of Repeat 1 sequence GTGAGCCACTGTGCCTGGCC (SEQ ID NO: 1) In the case when sequence motif is single directional, namely 3′ (as in Cas9) or 5′ (as in Cas12), the repeat sequence can be used for one direction motif identification and its reverse complement sequence could be used for another direction, such as Repeat_1 and Repeat_1R in Table 1. Other exemplary repeats in the genome, which can be used for spacer sequences targeting, are shown in Table 2. In some embodiments, the nucleic acid encoding the spacer sequence comprises any one of the sequences as set forth in SEQ ID NO: 1, 8-480, or their complementary sequences. In some embodiments, the spacer sequence comprises any one of the sequences as set forth in SEQ ID NO: 1, 8-480, or their complementary sequences, where the “T” is replaced by “U”. In some embodiments, for type V Cas nucleases with their PAM at the 5′ end of the spacer, such as FnCpf113 (Francisella novicida Cpf1), 5′-GGCCAGGCACAGTGGCTCAC-3′ can be directly used for PAM preference characterization. In some embodiments, for type II Cas nucleases with 3′ PAMs, such as SpCas9, SaCas9 or their variants, the reverse complementary sequence 5′-GTGAGCCACTGTGCCTGGCC-3′ can be used as the protospacer sequence (FIG. 1a).

Table 1. Oligonucleotides for targeting one human genome repeat (Repeat_1) and associated cloning vector-related sequences (overhang added for cloning) in one direction (Repeat_1_top and Repeat_1_bottom) for identification of 3′ PAM, its reverse direction (Repeat_1R_top and Repeat_1R_bottom) for identification of 5′ PAM, and one example variant spacer (Repeat_1V_top and Repeat_1V_bottom). “Y” indicates a C or T nucleotide. “R” indicates a G or A nucleotide.

| SEQ ID | Sequence name | Sequence (5′ to 3′) |
|---|---|---|
| 1 | Repeat_1 | GTGAGCCACTGTGCCTGGCC |
| 2 | Repeat_1_top | CACCGTGAGCCACTGTGCCTGGCC |
| 3 | Repeat_1_bottom | AAACGGCCAGGCACAGTGGCTCAC |
| 4 | Repeat_1R_top | CACCGGCCAGGCACAGTGGCTCAC |
| 5 | Repeat_1R_bottom | AAACGTGAGCCACTGTGCCTGGCC |
| 6 | Repeat_1V_top | CACCYGTGAGCCACTGTGCCTGGCC |
| 7 | Repeat_1V_bottom | AAACGGCCAGGCACAGTGGCTCACR |

Table 2. Other exemplary repeats in the genome.

| SEQ ID | Sequence (5′ to 3′) |
|---|---|
| 8 | GAGCCACCATGCCTGGCCAA |
| 9 | AAAAAAAAAAAAAAAAAAAC |
| 10 | AAAAAAAAAAAAAAAAAAAG |
| 11 | AAAAAAAAAAAAAAAAAAAT |
| 12 | AAAAAAAAAAAAAAAAAACA |
| 13 | AAAAAAAAAAAAAAAAAACC |
| 14 | AAAAAAAAAAAAAAAAAACT |
| 15 | AAAAAAAAAAAAAAAAAAGC |
| 16 | AAAAAAAAAAAAAAAAAAGG |
| 17 | AAAAAAAAAAAAAAAAAAGT |
| 18 | AAAAAAAAAAAAAAAAAATC |
| 19 | AAAAAAAAAAAAAAAAAATG |
| 20 | AAAAAAAAAAAAAAAAAATT |
| 21 | AAAAAAAAAAAAAAAAAGAC |
| 22 | AAAAAAAAAAAAAAAAAGAG |
| 23 | AAAAAAAAAAAAAAAAAGAT |
| 24 | AAAAAAAAAAAAAAAAAGCA |
| 25 | AAAAAAAAAAAAAAAAAGCC |
| 26 | AAAAAAAAAAAAAAAAAGCT |
| 27 | AAAAAAAAAAAAAAAAAGGA |
| 28 | AAAAAAAAAAAAAAAAAGGC |
| 29 | AAAAAAAAAAAAAAAAAGGG |
| 30 | AAAAAAAAAAAAAAAAAGGT |
| 31 | AAAAAAAAAAAAAAAAAGTA |
| 32 | AAAAAAAAAAAAAAAAAGTC |
| 33 | AAAAAAAAAAAAAAAAAGTG |
| 34 | AAAAAAAAAAAAAAAAAGTT |
| 35 | AAAAAAAAAAAAAAAAATAG |
| 36 | AAAAAAAAAAAAAAAAATCA |
| 37 | AAAAAAAAAAAAAAAAATTC |
| 38 | AAAAAAAAAAAAAAAAATTG |
| 39 | AAAAAAAAAAAAAAAAATTT |
| 40 | AAAAAAAAAAAAAAAAGAAC |
| 41 | AAAAAAAAAAAAAAAAGAAT |
| 42 | AAAAAAAAAAAAAAAAGACA |
| 43 | AAAAAAAAAAAAAAAAGAGT |
| 44 | AAAAAAAAAAAAAAAAGATA |
| 45 | AAAAAAAAAAAAAAAAGATT |
| 46 | AAAAAAAAAAAAAAAAGCAA |
| 47 | AAAAAAAAAAAAAAAAGCCA |
| 48 | AAAAAAAAAAAAAAAAGTAA |
| 49 | AAAAAAAAAAAAAAAATTAA |
| 50 | AAAAAAAAAAAAAAACAAAA |
| 51 | AAAAAAAAAAAAAAAGAAAT |
| 52 | AAAAAAAAAAAAAAAGAATA |
| 53 | AAAAAAAAAAAAAAAGAATT |
| 54 | AAAAAAAAAAAAAACAAAAA |
| 55 | AAAAAAAAAAAAACAAAAAA |
| 56 | AAAAAAAAAAAACAAAAAAA |
| 57 | AAAAAAACAAAACAAAACAA |
| 58 | AAAAAAAGAAAAAAAAAAAA |
| 59 | AAAAAAATAAATAAATAAAA |
| 60 | AAAAAAATACAAAAATTAGC |
| 61 | AAAAAACAAAAACAAAAACA |
| 62 | AAAAAACAAAACAAAACAAA |
| 63 | AAAAAACAAACAAACAAAAA |
| 64 | AAAAAACAAACAAACAAACA |
| 65 | AAAAAAGAAAAAAAAAAAAA |
| 66 | AAAAAATAAAATAAAATAAA |
| 67 | AAAAAATAAATAAATAAATA |
| 68 | AAAAACAAAACAAAACAAAA |
| 69 | AAAAACAAACAAACAAAAAA |
| 70 | AAAAACAAACAAACAAACAA |
| 71 | AAAAAGAAAAAAAAAAAAAA |
| 72 | AAAAATAAAATAAAATAAAA |
| 73 | AAAAATAAATAAATAAATAA |
| 74 | AAAAATAATAATAATAATAA |
| 75 | AAAACAAAAACAAAAACAAA |
| 76 | AAAACAAAACAAAACAAAAA |
| 77 | AAAACAAACAAACAAAAAAA |
| 78 | AAAACAAACAAACAAACAAA |
| 79 | AAAACAACAACAACAACAAC |
| 80 | AAAATAAATAAATAAATAAA |
| 81 | AAAATAATAATAATAATAAT |
| 82 | AAACAAAACAAAACAAAAAA |
| 83 | AAACAAACAAACAAAAAAAA |
| 84 | AAACAAACAAACAAAAAAAC |
| 85 | AAACAAACAAACAAACAAAA |
| 86 | AAACAACAACAACAACAACA |
| 87 | AAACACACACACACACACAC |
| 88 | AAATAATAATAATAATAATA |
| 89 | AAATTTTTTTTTTTTTTTTT |
| 90 | AACAAAACAAAACAAAAAAA |
| 91 | AACAAACAAACAAACAAAAA |
| 92 | AACAACAACAACAACAAAAA |
| 93 | AACACACACACACACACACA |
| 94 | AACTTTTTTTTTTTTTTTTT |
| 95 | AATACACACACACACACACA |
| 96 | AATCTTTTTTTTTTTTTTTT |
| 97 | AATTCTTTTTTTTTTTTTTT |
| 98 | AATTTTTTTTTTTTTTTTTG |
| 99 | AATTTTTTTTTTTTTTTTTT |
| 100 | ACAAAAAAAAAAAAAAAAAA |
| 101 | ACAAACAAACAAACAAAAAA |
| 102 | ACAACAACAACAACAACAAA |
| 103 | ACACACACACACACACACAA |
| 104 | ACACACACACACACACACAG |
| 105 | ACACACACACACACACACCC |
| 106 | ACCAAAAAAAAAAAAAAAAA |
| 107 | ACCTTTTTTTTTTTTTTTTT |
| 108 | ACTCTTTTTTTTTTTTTTTT |
| 109 | ACTTTTTTTTTTTTTTTTTT |
| 110 | AGAAAAAAAAAAAAAAAAAA |
| 111 | AGCAAAAAAAAAAAAAAAAA |
| 112 | AGCTTTTTTTTTTTTTTTTT |
| 113 | AGGCCAGGCACGGTGGCTCA |
| 114 | AGGCCAGGCGTGGTGGCTCA |
| 115 | AGGCCAGGTGTGGTGGCTCA |
| 116 | AGGCCGGGCACGGTGGCTCA |
| 117 | AGGCTGGGCACAGTGGCTCA |
| 118 | AGGCTGGGCACGGTGGCTCA |
| 119 | AGGCTGGGCATGGTGGCTCA |
| 120 | AGGCTGGGTGCAGTGGCTCA |
| 121 | AGGCTGGGTGTGGTGGCTCA |
| 122 | AGTGTGTGTGTGTGTGTGTG |
| 123 | AGTTTTTTTTTTTTTTTTTT |
| 124 | ATAATAATAATAATAATAAA |
| 125 | ATATATATATATATATATAA |
| 126 | ATATGGTTTGGCTGTGTCCC |
| 127 | ATCTTTTTTTTTTTTTTTTT |
| 128 | ATGAGCCACCATGCCCAGCC |
| 129 | ATGAGCCACCATGCCTGGCC |
| 130 | ATGAGCCACCGCGCCCGGCC |
| 131 | ATGAGCCACCGTGCCTGGCC |
| 132 | ATGAGCCACTGCACCTGGCC |
| 133 | ATGAGCCACTGTGCCCAGCC |
| 134 | ATGAGCCACTGTGCCTGGCC |
| 135 | ATTAAAAAAAAAAAAAAAAA |
| 136 | ATTATTATTATTATTATTTT |
| 137 | ATTCTTTTTTTTTTTTTTTT |
| 138 | ATTTCTTTTTTTTTTTTTTT |
| 139 | ATTTTTTTTTTTTTTGAGAC |
| 140 | ATTTTTTTTTTTTTTTGAGA |
| 141 | ATTTTTTTTTTTTTTTTGAG |
| 142 | ATTTTTTTTTTTTTTTTTGA |
| 143 | ATTTTTTTTTTTTTTTTTTG |
| 144 | ATTTTTTTTTTTTTTTTTTT |
| 145 | CAAAAAAAAAAAAAAAAAAT |
| 146 | CAAAAAAAAAAAAAAAAATT |
| 147 | CAAACAAACAAACAAAAAAA |
| 148 | CAAACAAACAAACAAAAAAC |
| 149 | CAACAACAACAACAACAAAA |
| 150 | CAATTTTTTTTTTTTTTTTT |
| 151 | CACACACACACACACACAAA |
| 152 | CACACACACACACACACACC |
| 153 | CACACACACACACACACACG |
| 154 | CACACACACACACACACACT |
| 155 | CACTTTTTTTTTTTTTTTTT |
| 156 | CATGAGCCACCACACCTGGC |
| 157 | CATGAGCCACCATGCCCAGC |
| 158 | CATGAGCCACCATGCCTGGC |
| 159 | CATGAGCCACCGCGCCCGGC |
| 160 | CATGAGCCACCGTGCCTGGC |
| 161 | CATGAGCCACTGCACCCAGC |
| 162 | CATGAGCCACTGCACCTGGC |
| 163 | CATGAGCCACTGTGCCCAGC |
| 164 | CATGAGCCACTGTGCCTGGC |
| 165 | CATTTTTTTTTTTTTTTTTT |
| 166 | CCAAAAAAAAAAAAAAAAAA |
| 167 | CCACACACACACACACACAC |
| 168 | CCAGGCACAGTGGCTCACAC |
| 169 | CCAGGCACAGTGGCTCACGC |
| 170 | CCAGGCACAGTGGCTCATGC |
| 171 | CCAGGCACGGTGGCTCACGC |
| 172 | CCAGGCACGGTGGCTCATGC |
| 173 | CCAGGCATGGTGGCTCACAC |
| 174 | CCAGGCATGGTGGCTCACGC |
| 175 | CCAGGCGCAGTGGCTCACGC |
| 176 | CCAGGCGCGGTGGCTCACGC |
| 177 | CCAGGCGTGGTGGCTCACGC |
| 178 | CCAGGTGCAGTGGCTCACAC |
| 179 | CCAGGTGCAGTGGCTCACGC |
| 180 | CCAGGTGCAGTGGCTCATGC |
| 181 | CCAGGTGCGGTGGCTCACGC |
| 182 | CCCTTTTTTTTTTTTTTTTT |
| 183 | CCGGGCACGGTGGCTCACGC |
| 184 | CCGGGCGCAGTGGCTCACGC |
| 185 | CCGGGCGCGGTGGCTCACGC |
| 186 | CCGGGCGCGGTGGCTCATGC |
| 187 | CCGGGCGTGGTGGCTCACGC |
| 188 | CCGGGTGCGGTGGCTCACGC |
| 189 | CCTTTTTTTTTTTTTTTTTT |
| 190 | CGTGAGCCACCACGCCCAGC |
| 191 | CGTGAGCCACCACGCCCGGC |
| 192 | CGTGAGCCACCACGCCTGGC |
| 193 | CGTGAGCCACCATGCCCAGC |
| 194 | CGTGAGCCACCATGCCTGGC |
| 195 | CGTGAGCCACCGCACCCGGC |
| 196 | CGTGAGCCACCGCACCTGGC |
| 197 | CGTGAGCCACCGCGCCCAGC |
| 198 | CGTGAGCCACCGCGCCCGGC |
| 199 | CGTGAGCCACCGCGCCTGGC |
| 200 | CGTGAGCCACCGTGCCCAGC |
| 201 | CGTGAGCCACCGTGCCCGGC |
| 202 | CGTGAGCCACCGTGCCTGGC |
| 203 | CGTGAGCCACTGCACCTGGC |
| 204 | CGTGAGCCACTGCGCCCAGC |
| 205 | CGTGAGCCACTGCGCCCGGC |
| 206 | CGTGAGCCACTGCGCCTGGC |
| 207 | CGTGAGCCACTGTGCCCAGC |
| 208 | CGTGAGCCACTGTGCCTGGC |
| 209 | CGTGTGTGTGTGTGTGTGTG |
| 210 | CTATTTTTTTTTTTTTTTTT |
| 211 | CTCAAAAAAAAAAAAAAAAT |
| 212 | CTCTTTTTTTTTTTTTTTTT |
| 213 | CTGGGCACAGTGGCTCACGC |
| 214 | CTGGGCACAGTGGCTCATGC |
| 215 | CTGGGCACGGTGGCTCACGC |
| 216 | CTGGGCACGGTGGCTCATGC |
| 217 | CTGGGCATGGTGGCTCACAC |
| 218 | CTGGGCATGGTGGCTCACGC |
| 219 | CTGGGCATGGTGGCTCATGC |
| 220 | CTGGGCGCAGTGGCTCACGC |
| 221 | CTGGGCGCGGTGGCTCACGC |
| 222 | CTGGGCGTGGTGGCTCACGC |
| 223 | CTGGGTGCAGTGGCTCATGC |
| 224 | CTGGGTGTGGTGGCTCATGC |
| 225 | CTGTGTGTGTGTGTGTGTGT |
| 226 | CTTTTTTTTTTTTTTTTTTA |
| 227 | CTTTTTTTTTTTTTTTTTTT |
| 228 | GAAAAAAAAAAAAAAAAAAA |
| 229 | GAATTTTTTTTTTTTTTTTT |
| 230 | GACACAGCCAAACCATATCA |
| 231 | GACTTTTTTTTTTTTTTTTT |
| 232 | GATATGGTTTGGCTGTGTCC |
| 233 | GATTTTTTTTTTTTTTTTTT |
| 234 | GCAAAAAAAAAAAAAAAAAA |
| 235 | GCATGAGCCACCACACCCAG |
| 236 | GCATGAGCCACCATGCCCAG |
| 237 | GCATGAGCCACCGCGCCCGG |
| 238 | GCATGAGCCACCGTGCCCAG |
| 239 | GCATGAGCCACCGTGCCTGG |
| 240 | GCATGAGCCACTGCACCCAG |
| 241 | GCATGAGCCACTGCACCTGG |
| 242 | GCATGAGCCACTGTGCCCAG |
| 243 | GCATGAGCCACTGTGCCTGG |
| 244 | GCCAGGCACAGTGGCTCACA |
| 245 | GCCAGGCACAGTGGCTCACG |
| 246 | GCCAGGCACAGTGGCTCATG |
| 247 | GCCAGGCACGGTGGCTCACG |
| 248 | GCCAGGCACGGTGGCTCATG |
| 249 | GCCAGGCATGGTGGCTCACA |
| 250 | GCCAGGCATGGTGGCTCACG |
| 251 | GCCAGGCATGGTGGCTCATG |
| 252 | GCCAGGCGCAGTGGCTCACG |
| 253 | GCCAGGCGCGGTGGCTCACG |
| 254 | GCCAGGCGTGGTGGCTCACG |
| 255 | GCCAGGTGCAGTGGCTCACA |
| 256 | GCCAGGTGCAGTGGCTCACG |
| 257 | GCCAGGTGCAGTGGCTCATG |
| 258 | GCCAGGTGCGGTGGCTCACG |
| 259 | GCCAGGTGTGGTGGCTCATG |
| 260 | GCCGGGCACGGTGGCTCACG |
| 261 | GCCGGGCGCAGTGGCTCACG |
| 262 | GCCGGGCGCGGTGGCTCACG |
| 263 | GCCGGGCGCGGTGGCTCATG |
| 264 | GCCGGGCGTGGTGGCTCACG |
| 265 | GCCGGGTGCGGTGGCTCACG |
| 266 | GCCTTTTTTTTTTTTTTTTT |
| 267 | GCGTGAGCCACCACGCCCAG |
| 268 | GCGTGAGCCACCACGCCCGG |
| 269 | GCGTGAGCCACCACGCCTGG |
| 270 | GCGTGAGCCACCATGCCCAG |
| 271 | GCGTGAGCCACCATGCCTGG |
| 272 | GCGTGAGCCACCGCACCCGG |
| 273 | GCGTGAGCCACCGCACCTGG |
| 274 | GCGTGAGCCACCGCGCCCAG |
| 275 | GCGTGAGCCACCGCGCCCGG |
| 276 | GCGTGAGCCACCGCGCCTGG |
| 277 | GCGTGAGCCACCGTGCCCAG |
| 278 | GCGTGAGCCACCGTGCCCGG |
| 279 | GCGTGAGCCACCGTGCCTGG |
| 280 | GCGTGAGCCACTGCACCTGG |
| 281 | GCGTGAGCCACTGCGCCCAG |
| 282 | GCGTGAGCCACTGCGCCCGG |
| 283 | GCGTGAGCCACTGCGCCTGG |
| 284 | GCGTGAGCCACTGTGCCCAG |
| 285 | GCGTGAGCCACTGTGCCTGG |
| 286 | GCTAATTTTTGTATTTTTTT |
| 287 | GCTGGGCACAGTGGCTCACG |
| 288 | GCTGGGCACAGTGGCTCATG |
| 289 | GCTGGGCACGGTGGCTCACG |
| 290 | GCTGGGCATGGTGGCTCACA |
| 291 | GCTGGGCATGGTGGCTCACG |
| 292 | GCTGGGCATGGTGGCTCATG |
| 293 | GCTGGGCGCAGTGGCTCACG |
| 294 | GCTGGGCGCGGTGGCTCACG |
| 295 | GCTGGGCGTGGTGGCTCACG |
| 296 | GCTGGGTGCAGTGGCTCATG |
| 297 | GCTTTTTTTTTTTTTTTTTT |
| 298 | GGAAAAAAAAAAAAAAAAAA |
| 299 | GGACACAGCCAAACCATATC |
| 300 | GGCCAGGCACAGTGGCTCAC |
| 301 | GGCCAGGCACAGTGGCTCAT |
| 302 | GGCCAGGCACGGTGGCTCAC |
| 303 | GGCCAGGCACGGTGGCTCAT |
| 304 | GGCCAGGCATGGTGGCTCAC |
| 305 | GGCCAGGCATGGTGGCTCAT |
| 306 | GGCCAGGCGCAGTGGCTCAC |
| 307 | GGCCAGGCGCGGTGGCTCAC |
| 308 | GGCCAGGCGTGGTGGCTCAC |
| 309 | GGCCAGGTGCAGTGGCTCAC |
| 310 | GGCCAGGTGCAGTGGCTCAT |
| 311 | GGCCAGGTGCGGTGGCTCAC |
| 312 | GGCCAGGTGTGGTGGCTCAC |
| 313 | GGCCGGGCACAGTGGCTCAC |
| 314 | GGCCGGGCACGGTGGCTCAC |
| 315 | GGCCGGGCATGGTGGCTCAC |
| 316 | GGCCGGGCGCAGTGGCTCAC |
| 317 | GGCCGGGCGCGGTGGCTCAC |
| 318 | GGCCGGGCGCGGTGGCTCAT |
| 319 | GGCCGGGCGTGGTGGCTCAC |
| 320 | GGCCGGGTGCAGTGGCTCAC |
| 321 | GGCCGGGTGCGGTGGCTCAC |
| 322 | GGCCGGGTGTGGTGGCTCAC |
| 323 | GGCTGGGCACAGTGGCTCAC |
| 324 | GGCTGGGCACAGTGGCTCAT |
| 325 | GGCTGGGCACGGTGGCTCAC |
| 326 | GGCTGGGCATGGTGGCTCAC |
| 327 | GGCTGGGCATGGTGGCTCAT |
| 328 | GGCTGGGCGCAGTGGCTCAC |
| 329 | GGCTGGGCGCGGTGGCTCAC |
| 330 | GGCTGGGCGTGGTGGCTCAC |
| 331 | GGCTGGGTGCAGTGGCTCAC |
| 332 | GGCTGGGTGCGGTGGCTCAC |
| 333 | GGCTGGGTGTGGTGGCTCAC |
| 334 | GGCTTTTTTTTTTTTTTTTT |
| 335 | GGGACACAGCCAAACCATAT |
| 336 | GGGCCGGGCGCGGTGGCTCA |
| 337 | GGGTGTGTGTGTGTGTGTGT |
| 338 | GGTGTGTGTGTGTGTGTGTG |
| 339 | GGTTTTTTTTTTTTTTTTTT |
| 340 | GTAAAAAAAAAAAAAAAAAA |
| 341 | GTCTCAAAAAAAAAAAAAAT |
| 342 | GTCTTTTTTTTTTTTTTTTT |
| 343 | GTGAGCCACCACACCCAGCC |
| 344 | GTGAGCCACCACACCCGGCC |
| 345 | GTGAGCCACCACACCTGGCC |
| 346 | GTGAGCCACCACGCCCAGCC |
| 347 | GTGAGCCACCACGCCCGGCC |
| 348 | GTGAGCCACCACGCCTGGCC |
| 349 | GTGAGCCACCATGCCCAGCC |
| 350 | GTGAGCCACCATGCCCGGCC |
| 351 | GTGAGCCACCATGCCTGGCC |
| 352 | GTGAGCCACCGCACCCAGCC |
| 353 | GTGAGCCACCGCACCCGGCC |
| 354 | GTGAGCCACCGCACCTGGCC |
| 355 | GTGAGCCACCGCGCCCAGCC |
| 356 | GTGAGCCACCGCGCCCGGCC |
| 357 | GTGAGCCACCGCGCCTGGCC |
| 358 | GTGAGCCACCGTGCCCAGCC |
| 359 | GTGAGCCACCGTGCCCGGCC |
| 360 | GTGAGCCACCGTGCCTGGCC |
| 361 | GTGAGCCACTGCACCCAGCC |
| 362 | GTGAGCCACTGCACCCGGCC |
| 363 | GTGAGCCACTGCACCTGGCC |
| 364 | GTGAGCCACTGCGCCCAGCC |
| 365 | GTGAGCCACTGCGCCCGGCC |
| 366 | GTGAGCCACTGCGCCTGGCC |
| 367 | GTGAGCCACTGTGCCCAGCC |
| 368 | GTGAGCCACTGTGCCCGGCC |
| 369 | GTGAGCCACTGTGCCTGGCC |
| 370 | GTGTGAGCCACCATGCCCAG |
| 371 | GTGTGAGCCACCATGCCTGG |
| 372 | GTGTGAGCCACTGCACCTGG |
| 373 | GTGTGAGCCACTGTGCCTGG |
| 374 | GTGTGTGTGTGTGTGTGTGG |
| 375 | GTGTGTGTGTGTGTGTGTTT |
| 376 | GTTCTTTTTTTTTTTTTTTT |
| 377 | GTTGTTGTTGTTGTTGTTTT |
| 378 | GTTTTTTGTTTGTTTGTTTG |
| 379 | GTTTTTTTGTTTGTTTGTTT |
| 380 | GTTTTTTTTTTTTTTTTTTT |
| 381 | TAAAAAAAAAAAAAAAAAAA |
| 382 | TAAAAAAAAAAAAAAAAAAG |
| 383 | TACTTTTTTTTTTTTTTTTT |
| 384 | TATATATATATATATATAAA |
| 385 | TATCTTTTTTTTTTTTTTTT |
| 386 | TATTATTATTATTATTATTT |
| 387 | TATTCTTTTTTTTTTTTTTT |
| 388 | TATTTATTTATTTATTTTTT |
| 389 | TCAAAAAAAAAAAAAAAAAT |
| 390 | TCCTTTTTTTTTTTTTTTTT |
| 391 | TCTCAAAAAAAAAAAAAAAT |
| 392 | TGAGCCACCACACCCAGCCT |
| 393 | TGAGCCACCACACCTGGCCT |
| 394 | TGAGCCACCACGCCTGGCCT |
| 395 | TGAGCCACCATGCCCAGCCA |
| 396 | TGAGCCACCATGCCCAGCCT |
| 397 | TGAGCCACCATGCCTGGCCA |
| 398 | TGAGCCACCGCGCCCGGCCA |
| 399 | TGAGCCACCGCGCCCGGCCC |
| 400 | TGAGCCACCGTGCCCAGCCT |
| 401 | TGAGCCACCGTGCCCGGCCT |
| 402 | TGAGCCACCGTGCCTGGCCT |
| 403 | TGAGCCACTGCACCCAGCCT |
| 404 | TGAGCCACTGTGCCCAGCCT |
| 405 | TGAGCCACTGTGCCTGGCCA |
| 406 | TGATATGGTTTGGCTGTGTC |
| 407 | TGATTTTTTTTTTTTTTTTT |
| 408 | TGCTTTTTTTTTTTTTTTTT |
| 409 | TGGCCAGGCACAGTGGCTCA |
| 410 | TGGCCAGGCATGGTGGCTCA |
| 411 | TGGCCGGGCGCGGTGGCTCA |
| 412 | TGGCTGGGCATGGTGGCTCA |
| 413 | TGGCTTTTTTTTTTTTTTTT |
| 414 | TGTCTTTTTTTTTTTTTTTT |
| 415 | TGTGAGCCACCATGCCCAGC |
| 416 | TGTGAGCCACCATGCCTGGC |
| 417 | TGTGAGCCACTGCACCTGGC |
| 418 | TGTGAGCCACTGTGCCTGGC |
| 419 | TGTGTGTGTGTGTGTGTATT |
| 420 | TGTGTGTGTGTGTGTGTGTT |
| 421 | TGTTGTTGTTGTTGTTGTTT |
| 422 | TGTTTGTTTGTTTGTTTTTT |
| 423 | TGTTTTTGTTTTTGTTTTTT |
| 424 | TGTTTTTTTTTTTTTTTTTT |
| 425 | TTAAAAAAAAAAAAAAAAAA |
| 426 | TTAATTTTTTTTTTTTTTTT |
| 427 | TTACTTTTTTTTTTTTTTTT |
| 428 | TTATATATATATATATATAT |
| 429 | TTATTATTATTATTATTTTT |
| 430 | TTATTTATTTATTTATTTTT |
| 431 | TTGCTTTTTTTTTTTTTTTT |
| 432 | TTGTGTGTGTGTGTGTGTGT |
| 433 | TTGTTTGTTTGTTTGTTTTT |
| 434 | TTGTTTTGTTTTGTTTTTTT |
| 435 | TTTAAAAAAAAAAAAAAAAA |
| 436 | TTTATATATATATATATATA |
| 437 | TTTATTATTATTATTATTAT |
| 438 | TTTATTTATTTATTTATTTT |
| 439 | TTTATTTTATTTTATTTTTT |
| 440 | TTTGTGTGTGTGTGTGTGTG |
| 441 | TTTGTTGTTGTTGTTGTTGT |
| 442 | TTTGTTTGTTTGTTTGTTTT |
| 443 | TTTGTTTTGTTTTGTTTTTT |
| 444 | TTTGTTTTTGTTTTTGTTTT |
| 445 | TTTTATTTATTTATTTTTTT |
| 446 | TTTTATTTTATTTTATTTTT |
| 447 | TTTTGTTGTTGTTGTTGTTG |
| 448 | TTTTGTTTGTTTGTTTGTTT |
| 449 | TTTTGTTTTGTTTTGTTTTT |
| 450 | TTTTGTTTTTTTTTTTTTTT |
| 451 | TTTTTGTTGTTGTTGTTGTT |
| 452 | TTTTTGTTTGTTTGTTTGTT |
| 453 | TTTTTGTTTGTTTGTTTTTT |
| 454 | TTTTTGTTTTGTTTTGTTTT |
| 455 | TTTTTGTTTTTTTTTTTTTT |
| 456 | TTTTTTGTTTGTTTGTTTGT |
| 457 | TTTTTTGTTTGTTTGTTTTT |
| 458 | TTTTTTGTTTTGTTTTGTTT |
| 459 | TTTTTTGTTTTTTTTTTTTT |
| 460 | TTTTTTTGTTTGTTTGTTTG |
| 461 | TTTTTTTGTTTGTTTGTTTT |
| 462 | TTTTTTTGTTTTGTTTTGTT |
| 463 | TTTTTTTGTTTTTTTTTTTT |
| 464 | TTTTTTTTGTTTGTTTGTTT |
| 465 | TTTTTTTTTTTTCTTTTTTT |
| 466 | TTTTTTTTTTTTTCTTTTTT |
| 467 | TTTTTTTTTTTTTTCTTTTT |
| 468 | TTTTTTTTTTTTTTTTTAAA |
| 469 | TTTTTTTTTTTTTTTTTAAT |
| 470 | TTTTTTTTTTTTTTTTTGCT |
| 471 | TTTTTTTTTTTTTTTTTGGT |
| 472 | TTTTTTTTTTTTTTTTTTAA |
| 473 | TTTTTTTTTTTTTTTTTTAC |
| 474 | TTTTTTTTTTTTTTTTTTCC |
| 475 | TTTTTTTTTTTTTTTTTTCT |
| 476 | TTTTTTTTTTTTTTTTTTGC |
| 477 | TTTTTTTTTTTTTTTTTTGG |
| 478 | TTTTTTTTTTTTTTTTTTGT |
| 479 | TTTTTTTTTTTTTTTTTTTA |
| 480 | TTTTTTTTTTTTTTTTTTTC |

### 5. The Polynucleotide, CRISPR-Cas System, Vector and Vector System

This disclosure also provides a polynucleotide for a specific application, wherein the polynucleotide comprises a spacer sequence or a nucleic acid sequence encoding such spacer sequence, wherein the spacer sequence is capable of binding to a repeat sequence from a genome of interest and the flanking sequences to the repeat are diversified; wherein the specific application is identification of adjacent sequence motif of a nuclease, identification of editing efficiency of a nuclease, or identification of off-target of a nuclease.

The terms “polynucleotide”, “nucleic acid”, or “oligonucleotide” refer to a polymer of nucleotides. The terms “polynucleotide”, “nucleic acid”, and “oligonucleotide”, may be used interchangeably. Typically, a polynucleotide comprises at least two nucleotides. DNAs and RNAs are polynucleotides.

In some embodiments, the polynucleotide is a gRNA comprising a spacer sequence or a nucleic acid sequence encoding such gRNA. In some embodiments, the gRNA further comprises a sequence capable of forming complex with the nuclease. In some embodiments, the gRNA further comprises a scaffold sequence. In some embodiments, the polynucleotide is a gRNA comprising a spacer sequence and a scaffold sequence. In some embodiments, the polynucleotide comprises a nucleic acid sequence encoding a gRNA comprising a spacer sequence and a scaffold sequence. In some embodiments, the gRNA is a dual-guide RNA (dgRNA). In some embodiments, the gRNA is a single-guide RNA (sgRNA). In some embodiments, the gRNA is a modified gRNA. In some embodiments, only one gRNA as disclosed in the present invention is configured to facilitate the identification or selection of protospacer adjacent motif (PAM) sequences recognized by variants of a Cas protein, such as those derived from SpCas9, thereby enabling efficient screen across a spectrum of Cas protein variants with diverse PAM compatibilities. In typical embodiments, the gRNA is capable of forming complex with the Cas protein. In some embodiments, the polynucleotide comprises a nucleic acid sequence encoding the gRNA thereof.

When we refer to “guide-RNA containing the repeat sequence or repeat's complementary sequence,” it is important to understand that the term “repeat sequence” or “repeat's complementary sequence” in this context represents an RNA sequence, where the nucleotide “T” is present as “U.” This does not directly represent the sequence form found in the genome, which could contain either “T” or “U” for the corresponding nucleotide in the “repeat sequence” or “repeat's complementary sequence”.

In some embodiments, the spacer sequence is complementary to at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 consecutive bases of the repeat sequence or the repeat's complementary sequence within the genome. In some embodiments, the repeat sequence has an occurrence of more than 4, 16, 64, 256, 1024, 4096, or 16384 times in the genome. In some embodiments, the repeat sequence has an occurrence of more than 4, 10, 16, 20, 30, 40, 50, 60, 70, 80, 90. 100, 110, 120, 130, 140, 150, 160, 170, 180,190, 200, 250, 300, 350, 400, 450, 500, 500, 600, 700, 800, 900, 100, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10000, 110000, 120000, 130000, 140000, 150000, 160000, 170000, 180000, 190000, 200000, 210000, 220000, 300000, 310000, 320000, 330000, 340000, 350000, 360000, 370000, 380000, 390000, 400000, 410000, 420000, 430000, 440000, 450000, 460000, 470000, 480000, 490000, 500000 times in the genome (any integer between the above-mentioned numbers should be considered as being included/documented in this disclosure). In some embodiments, the repeat sequence comprises any one of the sequences as set forth in SEQ ID NO: 1, 8-480. Further features of the repeat sequence disclosed in this invention are also encompassed within this section.

This disclosure also provides a CRISPR-Cas system comprising the polynucleotide herein. In some embodiments, the CRISPR-Cas system further comprises a Cas protein or a nucleic acid encoding the Cas protein. In some embodiments, the Cas Protein is selected from a Type II protein; optionally, the Type II protein is a Cas9, or its variants; preferably the Cas9 protein is Streptococcus pyogenes Cas9 (SpCas9), Staphylococcus aureus (SaCas9), or their variants. In some embodiments, the Cas protein is a Type V protein; preferably, the Type V protein is selected any one of Cas12a, Cas12b, Cas12c, Cas12d, Cas12e, Cas12f (also known as Cas14), Cas12g, Cas12h, Cas12i, Cas12j, Cas12k, or their variants. In some embodiments, the Cas protein is a Type VI protein; preferably, the Type VI protein is CasRx, C2c2, or their variants. Further features of the Cas protein disclosed in this invention are also encompassed within this section.

This disclosure also provides a vector comprising a nucleic acid encoding the polynucleotide herein. In some embodiments, the vector is a plasmid.

This disclosure also provides a vector system comprising a nucleic acid encoding the polynucleotide herein and a nucleic acid sequence encoding a Cas protein herein. In some embodiments, the Cas Protein is selected from a Type II protein; optionally, the Type II protein is a Cas9, or its variants; preferably the Cas9 protein is Streptococcus pyogenes Cas9 (SpCas9), Staphylococcus aureus (SaCas9), or their variants. In some embodiments, the Cas protein is a Type V protein; preferably, the Type V protein is selected any one of Cas12a, Cas12b, Cas12c, Cas12d, Cas12e, Cas12f (also known as Cas14), Cas12g, Cas12h, Cas12i, Cas12j, Cas12k, or their variants. In some embodiments, the Cas protein is a Type VI protein; preferably, the Type VI protein is CasRx, C2c2, or their variants. Further attributes of the Cas protein disclosed in this invention are also encompassed within this section. In some embodiments, the nucleic acid encoding the polynucleotide and the nucleic acid sequence encoding the Cas protein are placed in a same vector. In some embodiments, the nucleic acid encoding the polynucleotide and the nucleic acid sequence encoding the Cas protein are placed in different vectors. In some embodiments, the vector is a plasmid.

### EXAMPLES

Provided herein are examples that describe in more detail certain embodiments of the present disclosure. The examples provided herein are merely for illustrative purposes and are not meant to limit the scope of the invention in any way. All references given below and elsewhere in the present application are hereby included by reference.

Common materials and methods used in the examples.

### Identification of Repeat Sequences

The human genome (hg38) was used to calculate the frequencies of all 20-mer sequences by the jellyfish tools. Because a spacer starting with a G base at 5′ is required for most Cas nucleases, we selected all 20-mer sequences starting with a 5′ G. To avoid simple homopolymers and to increase base composition, we also excluded those 20-mers containing ‘AAA’, ‘TTT’, ‘CCC’, or ‘GGG’. Among the remaining 20-mer sequences, to retrieve their flanking sequences, we used BWA to map their chromosomal coordinates and retrieved 8 bases upstream and 8 bases downstream using samtools. The diversity of the flanking sequences of each of the 20-mer sequence was plotted using the ggseqlogo package.

### CRISPR/Cas Identification

Metagenomes were downloaded from EMBL-EBI MGnify, NCBI GenBank and the Joint Genome Institute, or assembled in-house using raw sequencing reads from NCBI Sequence Read Archive. We use a combinatorial pipeline that includes CCTyper, CRISPRcasIdentifier and OPFI to predict putative Cas proteins. MinCED was used to identify CRISPR arrays, any CRISPR arrays located adjacent to the predicted Cas, which typically comprises a CRISPR operon, were subjected to further analysis. Putative sequences of Cas were scanned by Interproscan to identify and annotate conserved domains. Selected Cas proteins were aligned with MAFFT, and a phylogenetic tree was constructed using FastTree2.

### Cell Culture

HEK293T (CRL3216, ATCC) and HepG2 (CRL11997, ATCC) cell lines were purchased from ATCC (American Type Culture Collection, VA, USA). HEK293T cells were cultured in Dulbecco's Modified Eagle Medium (C11995500BT, GIBCO) and HepG2 cells were cultured in Eagle's Minimum Essential Medium (ATCC 30-2003) supplemented with 10% fetal bovine serum (10270-106, GIBCO) and incubated at 37° C. with 5% CO2 in a constant temperature incubator. Cell passaging was performed at a 1:3 split ratio when the cells reached 90% confluence.

### Plasmids and Oligonucleotides

The plasmids used in these experiments were purchased from the non-profit plasmid repository Addgene (Watertown, MA, U.S.). The plasmid lentiCRISPRv2 (Addgene #52961) was used to express wild-type SpCas9; BPK2139 (Addgene #65776) to express wild-type SaCas9; pY004 (Addgene #69976) to express wild-type FnCpf1; pET-CjCas9 (Addgene #89754) to express wild-type CjCas9 and the plasmids BPK1520 (Addgene #65777), BPK2660 (Addgene #70709), pU6-Fn-crRNA (Addgene #78958), pU6-cj-E sgRNA (Addgene #169915) were used to express SpCas9 sgRNA, SaCas9 sgRNA, FnCpf1 crRNA and CjCas9 sgRNA, respectively. Oligonucleotide duplexes corresponding to the target spacer sequences were purchased from GENEWIZ (Suzhou, China).

### Plasmid Construction

Oligonucleotide duplexes corresponding to sgRNA sequences (paired top and bottom single-stranded oligos) were annealed together using the following program: 95° C., 3 min; 70 cycles of (95° C., 1 min with −1° C. per cycle); 4° C. holds. Annealed DNA segments were inserted into BsmbI digested sgRNA expression plasmids. After transformation into bacteria and selection, the plasmids were purified by PureLink™ HiPure Plasmid Midiprep Kit (Invitrogen). Sequences of guide-insertion in the plasmids were confirmed by Sanger sequencing (BGI, Hong Kong).

Double-Stranded Oligonucleotide (dsODN) Preparation

dsODN oligos were purchased from GENEWIZ with HPLC purification. Each oligo was resuspended in 1×TE buffer (Thermo Fisher 12090015) to a final concentration of 250 μM. These oligos were then annealed at 100 μM in 1× annealing buffer (10 mM Tris-HCl, 50 mM NaCl, 1 mM EDTA, pH7.4) on thermocycler. The program was 95° C., 3 min; 70×[95° C. 1 min (−1° C. per cycle)]; 4° C. holds.

### Cell Transfection

Guide RNAs and Cas proteins plasmids were transfected into cells using Lipofectamine™ 3000 Transfection Reagent (ThermoFisher, L3000015) following the manufacturer's instruction. Cells were cultured at the density of 1×105 per well in 24-well plate. For each well, 400 ng of gRNA and Cas expression plasmids together with 5 pmol of annealed dsODN were mixed with 1 μL of P3000 reagents in 25 μL of Opti-MEM medium, and then mixed with 1.5 μL of Lipofectamine 3000 Reagent in 25 μL of Opti-MEM medium to generate a total volume of 50 μL DNA-lipid complex, followed by incubation for 10 minutes at room temperature. The transfection complex was added into individual wells. The plate was maintained in a cell culture incubator for 48-72 hours.

### DNA Extraction

Genomic DNA was extracted using the MiniBEST Universal Genomic DNA Extraction Kit (TaKaRa) and quantified using Qubit™ dsDNA HS Assay Kit (Invitrogen) in Qubit 3.0 fluorometer.

### GUIDE-Seq

Genomic DNA was extracted 72 hours post-transfection and 500 ng of DNA was used for next-generation sequencing (NGS) library construction according to previous work with modifications. Briefly, the library preparation procedure contains fragmentation, end-repairation, A-tailing, adaptor ligation and two rounds of anchored hemi-nested PCR. The libraries were quantified with KAPA Library Quantification Kits and sequenced on NextSeq 500 System (Illumina, San Diego, CA, USA) using 150-cycle (2×75 bp paired-end) or 300-cycle kit (2×150 bp paired-end) with custom sequencing primers. Sequencing data (FASTQ files) were analyzed using the GUIDE-seq pipeline (from https://github.com/aryeelab/guideseq). The off targets were identified using the criteria of ≤5 mismatched bases with the intended targeting protospacer.

### Example 1. Identification of SpCas9 PAM Sequence Using Human Cell Line

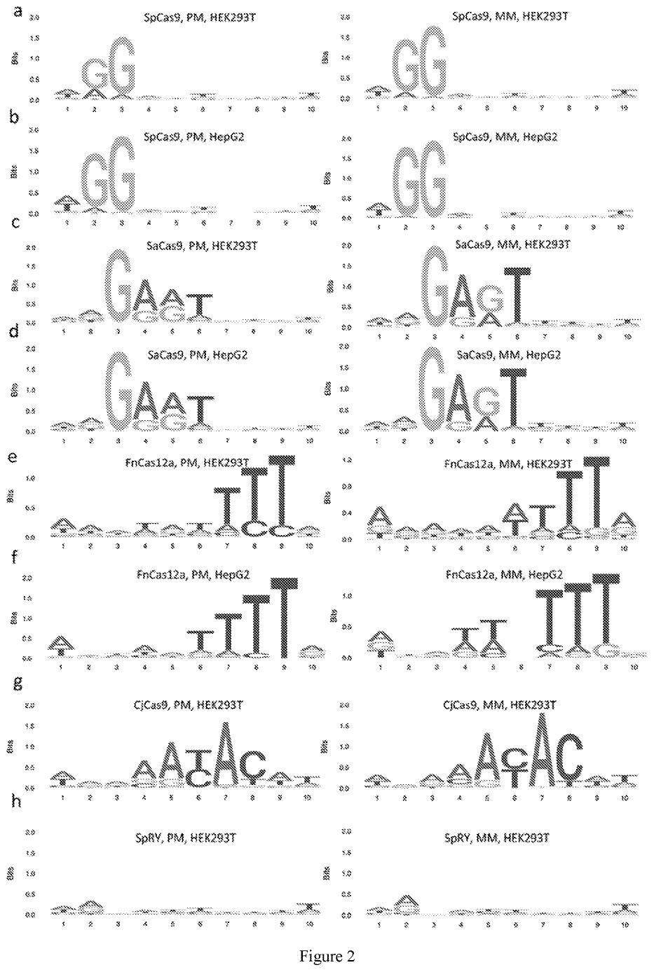

The Repeat 1 (SEQ IDs 2 and 3, Table 1) was used as the protospacer sequence to guide SpCas9 editing in HEK293T and HepG2 cells, respectively. SpCas9 has its PAM sequence at the 3′ of the protospacer or targeting sequence. The genome-wide edited sequences were analyzed by the GUIDE-seq method. Different from other PAM identification methods, this invention involves both on-target and off-target information. When the cleaved sequence derived from the GUIDE-Seq analysis is not identical to the intended targeting repeat sequence, namely the off-target sequences containing mismatched bases with the targeted sequence, we can still take advantage of these off-target sequences and infer the PAM sequences of the SpCas9 nuclease. The inferred PAM sequences were summarized into two categories, perfect matched (PM) and mismatched (MM) (FIGS. 2a and 2b). The PAM sequence inferred by using the method disclosed here was dominantly 5′-NGG-3′ (N=A or T or C or G) at the 3′ side of the protospacer (Repeat_1) for SpCas9, as expected.

### Example 2. Identification of SaCas9 PAM Sequence Using Human Cell Line

The Repeat 1 (SEQ IDs 2 and 3, Table 1) was also used as the protospacer sequence to guide another nuclease, SaCas9, for gene editing in HEK293T and HepG2 cells, respectively. The PAM sequence of SaCas9 is at the 3′ of the protospacer or targeting sequence. The genome-wide edited sequences were analyzed by the GUIDE-seq method. The inferred PAM sequences were summarized into two categories, perfect matched (PM) and mismatched (MM) (FIGS. 2c and 2d). The PAM sequence inferred by using the method disclosed here was dominantly 5′-NNGRRT (N=A or T or C or G, R=A or G)-3′ at the 3′ side of the protospacer (Repeat_1) for SaCas9, as expected.

### Example 3. Identification of FnCas12a PAM Sequence Using Human Cell Line

The Repeat 1 reverse sequence “Repeat_1R” (SEQ IDs 4 and 5, Table 1) was used as the protospacer sequence to guide another nuclease, FnCas12a, for editing in HEK293T and HepG2 cells, respectively. The PAM sequence of FnCas12a is at the 5′ of the protospacer or targeting sequence. The genome-wide edited sequences were analyzed by the GUIDE-seq method. The inferred PAM sequences were summarized into two categories, perfect matched (PM) and mismatched (MM) (FIGS. 2e and 2f). The PAM sequence inferred by using the method disclosed here was dominantly 5′-TTTN-3′ (N=A or T or C or G) at the 5′ side of the protospacer (Repeat_1R) for FnCas12a, as expected.

### Example 4. Identification of CjCas9 PAM Sequence Using Human Cell Line

The Repeat 1 with 1-nt extension “Repeat_1V” (SEQ IDs 6 and 7, Table 1) was also used as the protospacer sequence to guide CjCas9 editing in HEK293T cells. The PAM sequence of CjCas9 is at the 3′ of protospacer or targeting sequence. It remains a challenge to identify long PAM sequences, such as longer than 6 bases, in part due to the difficulties in maintaining the diversity of the large sequence libraries. To address this limitation, current methods attempt to identify long PAM are to divide long PAM sequence into two or more shorter parts and conduct sequential experiments to identify one after another shorter part, which is labor-intensive and costly. Here, leveraging the perfect and large sequence diversity that is naturally maintained in each single cell, as disclosed in this invention, we can easily identify, in one single experiment, the long PAM sequence dominantly 5′-NNNNRYAC-3′ (N=A or T or C or G, R=A or G, Y=C or T) at the 3′ side of the protospacer (Repeat_1) for CjCas9, consistent with previous stepwise and costly methods. The genome-wide edited sequences were analyzed by the GUIDE-seq method. The inferred PAM sequences were summarized into two categories, perfect matched (PM) and mismatched (MM) (FIG. 2g).

### Example 5. Identification of Cas Proteins with Minimal PAM Requirement Using Human Cell Line

The CRISPR-Cas system requires PAM recognition and limits the targetable sequences within a genome. A number of PAM-relaxing Cas nuclease variants have been developed to broaden the targeting range of the CRISPR-Cas system. In this case, negative selection-based methods would be incapable of identifying Cas nuclease with nearly random PAM preferences, i.e., PAM-less. We performed our methods to characterize a previously developed PAM-less Cas nuclease, SpRY, and identified the PAM sequence as 5′-NNNN-3′ (N=A or T or C or G) at the 3′ side of the protospacer (Repeat_1), demonstrating the capability of our methods in identification of nearly random PAM recognition (FIG. 2h).

### Example 6. Identification of Cas Proteins with Minimal PAM Requirement Using Human Cell Line

Well-established PAMs of SpCas9, SaCas9, and FnCpf1 validation experiments were also performed in three other cell lines, Huh7, and HeLa cell lines (supplementary FIG. S1). The results showed nearly identical PAM profiles as those using HEK 293T cells. Furthermore, we tested using another repeat sequence (Rep-2: 5′-GAGCCACCATGCCTGGCCAA-3′, SEQ ID NO: 8) that occurs 1,989 times in the human genome (3,978 occurrences in a human diploid cell) as the protospacer for analysis of SpCas9, SaCas9, and FnCpf1. The results were the same, namely NGG at 3′, NNGRRT at 3′, and TTTN at 5′ for the three nucleases, respectively.

### Example 7. PAM Assay for Novel Cas Discovery

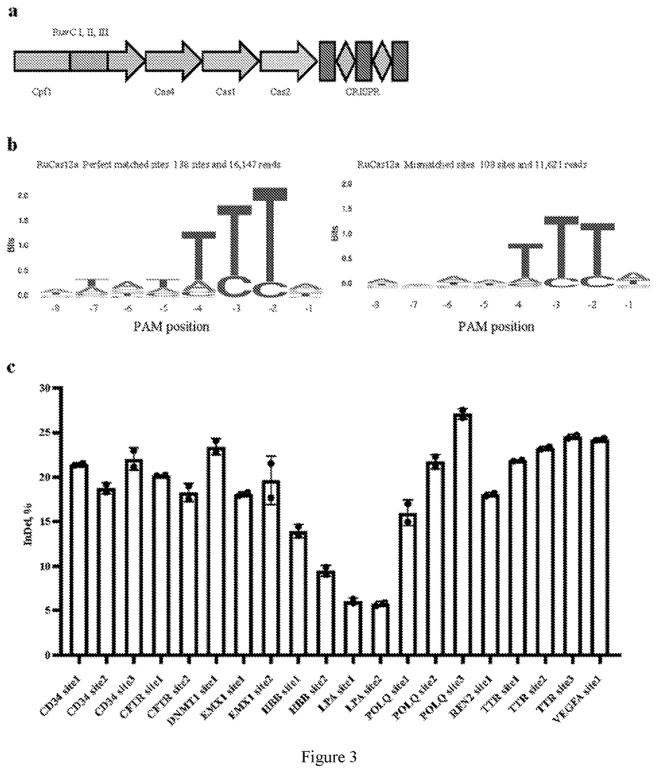

After establishing the simplicity and accuracy of the PAM assay in identifying various Cas nucleases, we sought to demonstrate its utility for novel Cas discovery. Using a metagenomics approach (see Methods), we identified one novel Type V-A CRISPR-Cas candidate derived from Ruminococcus Dsp902787825, named RuCas12a (FIG. 3a). We performed the GenomePAM assay using 5′-GGCCAGGCACAGTGGCTCAC-3′ as the protospacer to characterize its potential activity and 5′ PAM requirement. The result revealed that the PAM preference of RuCas12a was TTTN at its 5′ end (FIG. 3b). To further validate and comprehensively evaluate genome editing efficiency of RuCas12, we used 20 regular (non-repetitive) genomic sites containing a 5′ end ‘TTTG’ in human genes CD34, CFTR, DNMT1, EMX1, HBB, LPA, POLQ, RFN2, TTR and VEGFA. The editing efficiencies ranged from 5.5% to 26.7% across the 20 genomic loci in HEK293T cells (FIG. 3c).

### Example 8: Simultaneous Comparison of Genome-Wide Potency and Specificity of Different Cas9 Variants

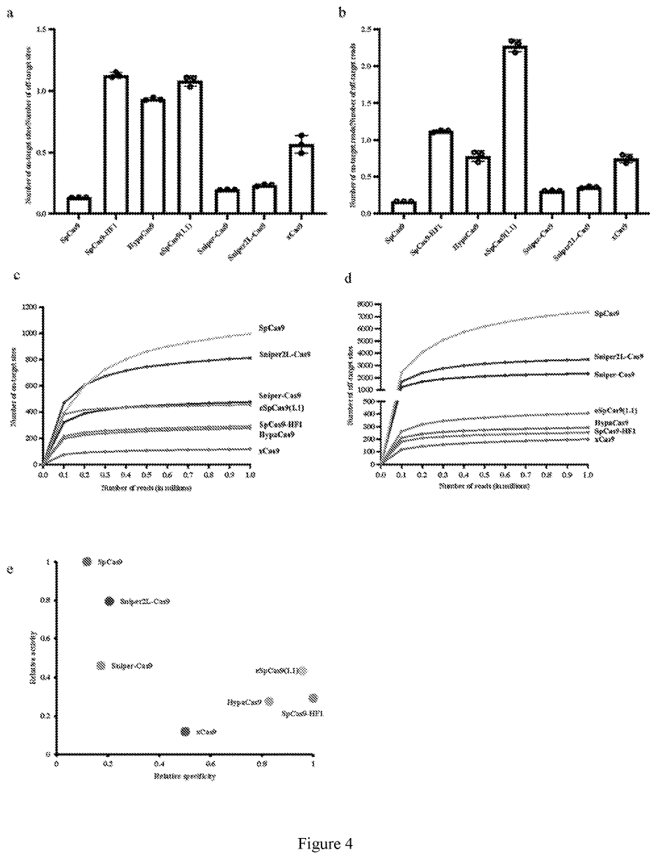

Many SpCas9 variants have been developed to reduce off-target effects, such as SpCas9-HF1, HypaCas9, eSpCas9 (1.1), Sniper-Cas9, and Sniper2L-Cas9 and broadened PAM compatibilities like xCas940. Oftentimes, dozens of gene loci are used to assess the fidelity and activity of Cas9 variants. It would be desirable to simultaneous evaluate Cas nuclease potency and specificity with less a laborious method than traditional library-based approaches1. A method based on large-scale synthetic oligos (n=26,891) containing targeting sequences and mismatched sequences has been developed for this purpose20. PAM assay in this disclosure uses a single protospacer oligo that provides thousands of perfect match sites in one human cell and, when integrated with the GUIDE-Seq method, also tens of thousands of potential off-target sites. Therefore, we sought to evaluate the feasibility of simultaneous comparing genome-wide potency and specificity of different Cas9 variants. We performed PAM assay experiments with 5′-GTGAGCCACTGTGCCTGGCC-3′ as the targeting protospacer for WT SpCas9 and seven variants in parallel with the same amounts of Cas and of sgRNA expression plasmids. The ratios of on-to-off target sites were highest for SpCas9-HF1 (mean 1.13), eSpCas9 (1.1) (mean 1.08), and HypaCas9 (mean 0.93), followed by xCas9 (mean 0.57), Sniper2L-Cas9 (mean 0.23) and Sniper-Cas9 (mean 0.20), and the lowest for WT SpCas9 (mean 0.13) (FIG. 4a). Similarly, the ratios of on-to-off target reads were 1.12, 2.28, 0.78, 0.75, 0.36, 0.31 and 0.17, respectively (FIG. 4b). To evaluate Cas9 cleavage dynamics with increasing probing data, we sampled the datasets from 100K up to 1M sequencing reads for GUIDE-Seq analysis. The number of on-target sites identified given the same amount of sequencing data (reflecting potency) increased from fast to slow as follows: WT~Sniper2L-SpCas9>>Sniper-SpCas9~eSpCas9 (1.1)~SpCas9-HF1~HypaCas9>xCas9 (FIG. 4c). The number of off-target sites identified given the same amount of data (reflecting specificity) increased from fast to slow as follows: xCas9~HypaCas9~SpCas9-HF1~eSpCas9 (1.1)>Sniper-SpCas9>Sniper2L-SpCas9>>WT (FIG. 4d).

To compare general activity and specificity of different SpCas9 variants in one place, we used the 1M-read datasets. We defined relative activity as the number of perfect match sites relative to the number of mismatched sites identified by WT SpCas9, and relative specificity as the ratio of perfect match to mismatched target site numbers relative to the ratio in SpCas9-HF1 (because SpCas-HF1 had the highest ratio among the seven SpCas9 tested here) (FIG. 4e) The scatter plot showed that WT and Sniper2L-SpCas9s were more potent but less specific than other variants, whereas eSpCas9, SpCas9-HF1 and HypaCas9 were more specific but less potent than WT SpCas9 (FIG. 4e).

### Example 9: Profiling Chromatin Accessibility in Different Human Cell Lines

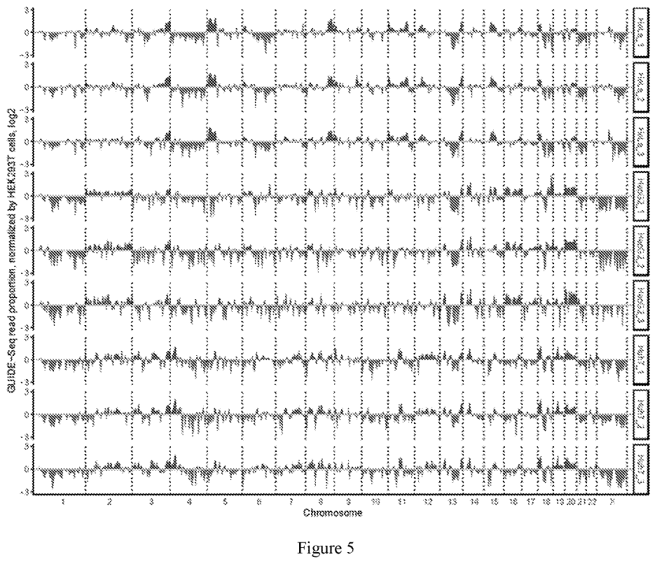

Chromatin conformation has been shown to affect Cas nuclease genome editing on relatively small numbers of genomic targets but not on the genome-wide scale. To assess genome-wide targeting profiles in different cell lines, we performed PAM assays using SpCas9 and 5′-GTGAGCCACTGTGCCTGGCC-3′ in HEK293T, HepG2, Huh7 and HeLa cell lines with triplicates (FIG. 5). Genome-wide chromatin accessibility was defined as the number of targeting reads per 5 M base chromosome window. Relative to HEK293T, the results from triplicates of the same cell line showed consistent and reproducible genome-wide chromatin profiles (FIG. 5). Interestingly, hepatocyte-derived cell lines HepG2 and Huh7 showed very similar chromatin accessibility profiles in contrast to that of HeLa cells, indicating similar chromatin accessibility of the same tissue (FIG. 5).

The exemplary embodiments of the present invention are thus fully described. Although the description referred to particular embodiments, it will be clear to one skilled in the art that the present invention may be practiced with variation of these specific details. The methods/steps discussed in one figure can be added to or exchanged with methods/steps in other figures. Hence this invention should not be construed as limited to the embodiments set forth herein.

What is claimed is:

1. A method for identification of adjacent sequence motif (adjacent motif) of a nuclease using a sequence that occurs more than once in a genome, wherein the method comprises the steps of:
(1) identifying a repeat sequence from a genome of interest, where the flanking sequences to the repeat sequence are diversified;
(2) contacting a nuclease of interest with the genome to target the repeat sequence; and
(3) analyzing the flanking sequences of modified or unmodified repeat sequence(s), so as to identify the adjacent sequence motif of the nuclease.

2. The method of claim 1, wherein the nuclease comprises a clustered regularly interspaced short palindromic repeats (CRISPR)-associated protein (Cas) and the adjacent motif is a protospacer adjacent motif (PAM).

3. The method of claim 1, wherein the nuclease in step (2) comprises a CRISPR-Cas protein (Cas protein), a guide-RNA containing the repeat sequence or repeat's complementary sequence is utilized to direct the Cas protein to target the repeat.

4. The method of claim 1, wherein the nuclease is in the form of DNA plasmid and is directly expressed in a living cell.

5. The method of claim 1, wherein the nuclease is in the form of mRNA and is translated in a living cell.

6. The method of any one of the preceding claims, wherein the genome of interest is a eukaryotic genome; optionally, the eukaryotic genome is from human, mouse, rat, pig, monkey, or other animals, plants, fungi, or protists.

7. The method of any one of the preceding claims, wherein the genome of interest is a prokaryotic genome; optionally, the prokaryotic genome is from bacterial or archaea.

8. The method of claim 2, wherein the Cas protein is a Type II protein; optionally, the Type II protein is a Cas9, or its variants; preferably the Cas9 protein is Streptococcus pyogenes Cas9 (SpCas9), Staphylococcus aureus (SaCas9), or their variants.

9. The method of claim 2, wherein the Cas protein is a Type V protein; preferably, the Type V protein is selected any one of Cas12a, Cas12b, Cas12c, Cas12d, Cas12e, Cas12f (also known as Cas14), Cas12g, Cas12h, Cas12i, Cas12j, Cas12k, or their variants.

10. The method of claim 2, wherein the Cas protein is a Type VI protein; preferably, the Type VI protein is CasRx, C2c2, or their variants.

11. The method of any one of claims 1 to 10, wherein the length of the PAM is 1 to 10 bases, 11 to 20 bases, or 21 to 100 bases.

12. The method of any one of claims 1 to 10, wherein the length of the PAM is 1, 2, 3, 4, 5, 6, 7, 8, 9, or 10 bases.

13. The method of any one of claims 1 to 12, wherein the PAM identified is used for CRISPR-based gene-editing therapeutic applications.

14. The method of any one of claims 1 to 12, wherein the PAM identified is used for nuclease-based molecular diagnosis applications.

15. The method of any one of claims 1 to 14, wherein the method further comprises the step of: quantifying the edited sequences with perfectly matched (on-targets) and with mismatched bases (off-targets) with the repeat sequence to infer, under given experiment conditions, the potency of the nuclease that is correlated with the number of edited loci in a genome and the total number of edited DNA fragments at the on-target and off-target loci.

16. The method of any one of claims 1 to 14, wherein the method further comprises the step of: quantifying the edited sequences with perfectly matched (on-targets) and with mismatched bases (off-targets) with the repeat sequence to infer, under given experiment conditions, the fidelity of the nuclease that is correlated with the proportion of edited on-target loci in the genome and the total number of edited DNA fragments at these on-target loci relative to the off-target loci and off-target DNA fragments, respectively.

17. A method for identification of editing efficiency of a nuclease using a sequence that occurs more than once in a genome, wherein the method comprises the steps of:
(1) identifying a repeat sequence from a genome of interest, where the flanking sequences to the repeat sequence are diversified;
(2) contacting a nuclease of interest with the genome to target the repeat sequence; and
(3) measuring the editing efficiency of the target loci.

18. A method for identification of off-target of a nuclease using a sequence that occurs more than once in a genome, wherein the method comprises the steps of:
(1) identifying a repeat sequence from a genome of interest, where the flanking sequences to the repeat sequence are diversified;
(2) contacting a nuclease of interest with the genome to target the repeat sequence; and
(3) quantifying the edited sequences with perfectly matched (on-targets) and with mismatched bases (off-targets).

19. The method of claim 17 or 18, wherein the nuclease comprises a clustered regularly interspaced short palindromic repeats (CRISPR)-associated protein (Cas).

20. The method of any one of claims 17-19, wherein the nuclease in step (2) comprises a CRISPR-Cas protein (Cas protein), a guide-RNA containing the repeat sequence or repeat's complementary sequence is utilized to direct the Cas protein to target the repeat.

21. The method of any one of claims 17-20, wherein the nuclease is in the form of DNA plasmid and is directly expressed in a living cell.

22. The method of any one of claims 17-21, wherein the nuclease is in the form of mRNA and is translated in a living cell.

23. The method of any one of the preceding claims 17-22, wherein the genome of interest is a eukaryotic genome; optionally, the eukaryotic genome is from human, mouse, rat, pig, monkey, or other animals, plants, fungi, or protists.

24. The method of any one of the preceding claims 17-23, wherein the genome of interest is a prokaryotic genome; optionally, the prokaryotic genome is from bacterial or archaea.

25. The method of any one of claims 19-24, wherein the Cas protein is a Type II protein; optionally, the Type II protein is a Cas9, or its variants; preferably the Cas9 protein is Streptococcus pyogenes Cas9 (SpCas9), Staphylococcus aureus (SaCas9), or their variants.

26. The method of any one of claims 19-25, wherein the Cas protein is a Type V protein; preferably, the Type V protein is selected any one of Cas12a, Cas12b, Cas12c, Cas12d, Cas12e, Cas12f (also known as Cas14), Cas12g, Cas12h, Cas12i, Cas12j, Cas12k, or their variants.

27. The method of any one of claims 19-26, wherein the Cas protein is a Type VI protein; preferably, the Type VI protein is CasRx, C2c2, or their variants.

28. A polynucleotide for a specific application, wherein the polynucleotide comprises a spacer sequence or a nucleic acid sequence encoding such spacer sequence, wherein the spacer sequence is capable of binding to a repeat sequence or a repeat's complementary sequence from a genome of interest and the flanking sequences to the repeat sequence are diversified; and wherein the specific application is identification of adjacent sequence motif of a nuclease, identification of editing efficiency of a nuclease, or identification of off-target of a nuclease.

29. The polynucleotide of claim 28, wherein the spacer sequence is complementary to at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 consecutive bases of the repeat sequence or repeat's complementary sequence within the genome.

30. The polynucleotide of claim 28 or 29, wherein the repeat sequence has an occurrence of more than 4, 16, 64, 256, 1024, 4096, or 16384 times in the genome.

31. The polynucleotide of any one of claims 28-30, wherein the repeat sequence comprises any one of the sequences as set forth in SEQ ID NO: 1, 8-480.

32. A CRISPR-Cas system comprising the polynucleotide of any one of claims 28-31.

33. The CRISPR-Cas system of claim 32, wherein the CRISPR-Cas system further comprises a Cas protein or a nucleic acid encoding the Cas Protein thereof.

34. The CRISPR-Cas system of claim 32 or 33, wherein the Cas Protein is selected from a Type II protein; optionally, the Type II protein is a Cas9, or its variants; preferably the Cas9 protein is Streptococcus pyogenes Cas9 (SpCas9), Staphylococcus aureus (SaCas9), or their variants.

35. The CRISPR-Cas system of claim 32 or 33, wherein the Cas protein is a Type V protein; preferably, the Type V protein is selected any one of Cas12a, Cas12b, Cas12c, Cas12d, Cas12e, Cas12f (also known as Cas14), Cas12g, Cas12h, Cas12i, Cas12j, Cas12k, or their variants.

36. The CRISPR-Cas system of claim 32 or 33, wherein the Cas protein is a Type VI protein; preferably, the Type VI protein is CasRx, C2c2, or their variants.

37. A vector comprising a nucleic acid encoding the polynucleotide of any one of the claims 28-31.

38. A vector system comprising a nucleic acid encoding the polynucleotide of any one of the claims 28-31 and a nucleic acid sequence encoding a Cas protein.

39. The vector system of claim 37, wherein the nucleic acid encoding the polynucleotide of any one of the claims 28-31 and the nucleic acid sequence encoding the Cas protein are placed in a same vector.

40. The vector system of claim 37, wherein the nucleic acid encoding the polynucleotide of any one of the claims 28-31 and the nucleic acid sequence encoding the Cas protein are placed in different vectors.


SEQUENCE LISTING (480 sequences)

SEQ ID NO: 1 - DNA, 20 bp, synthetic construct
  source 1..20: mol_type = other DNA
gtgagccactgtgcctggcc

SEQ ID NO: 2 - DNA, 24 bp, synthetic construct
  source 1..24: mol_type = other DNA
caccgtgagccactgtgcctggcc

SEQ ID NO: 3 - DNA, 24 bp, synthetic construct
  source 1..24: mol_type = other DNA
aaacggccaggcacagtggctcac

SEQ ID NO: 4 - DNA, 24 bp, synthetic construct
  source 1..24: mol_type = other DNA
caccggccaggcacagtggctcac

SEQ ID NO: 5 - DNA, 24 bp, synthetic construct
  source 1..24: mol_type = other DNA
aaacgtgagccactgtgcctggcc

SEQ ID NO: 6 - DNA, 25 bp, synthetic construct
  source 1..25: mol_type = other DNA
caccygtgagccactgtgcctggcc

SEQ ID NO: 7 - DNA, 25 bp, synthetic construct
  source 1..25: mol_type = other DNA
aaacggccaggcacagtggctcacr

SEQ ID NO: 8 - DNA, 20 bp, synthetic construct
  source 1..20: mol_type = other DNA
gagccaccatgcctggccaa

SEQ ID NO: 9 - DNA, 20 bp, synthetic construct
  source 1..20: mol_type = other DNA
aaaaaaaaaaaaaaaaaaac

SEQ ID NO: 10 - DNA, 20 bp, synthetic construct
  source 1..20: mol_type = other DNA
aaaaaaaaaaaaaaaaaaag

SEQ ID NO: 11 - DNA, 20 bp, synthetic construct
  source 1..20: mol_type = other DNA
aaaaaaaaaaaaaaaaaaat

SEQ ID NO: 12 - DNA, 20 bp, synthetic construct
  source 1..20: mol_type = other DNA
aaaaaaaaaaaaaaaaaaca

SEQ ID NO: 13 - DNA, 20 bp, synthetic construct
  source 1..20: mol_type = other DNA
aaaaaaaaaaaaaaaaaacc

SEQ ID NO: 14 - DNA, 20 bp, synthetic construct
  source 1..20: mol_type = other DNA
aaaaaaaaaaaaaaaaaact

SEQ ID NO: 15 - DNA, 20 bp, synthetic construct
  source 1..20: mol_type = other DNA
aaaaaaaaaaaaaaaaaagc

SEQ ID NO: 16 - DNA, 20 bp, synthetic construct
  source 1..20: mol_type = other DNA
aaaaaaaaaaaaaaaaaagg

SEQ ID NO: 17 - DNA, 20 bp, synthetic construct
  source 1..20: mol_type = other DNA
aaaaaaaaaaaaaaaaaagt

SEQ ID NO: 18 - DNA, 20 bp, synthetic construct
  source 1..20: mol_type = other DNA
aaaaaaaaaaaaaaaaaatc

SEQ ID NO: 19 - DNA, 20 bp, synthetic construct
  source 1..20: mol_type = other DNA
aaaaaaaaaaaaaaaaaatg

SEQ ID NO: 20 - DNA, 20 bp, synthetic construct
  source 1..20: mol_type = other DNA
aaaaaaaaaaaaaaaaaatt

SEQ ID NO: 21 - DNA, 20 bp, synthetic construct
  source 1..20: mol_type = other DNA
aaaaaaaaaaaaaaaaagac

SEQ ID NO: 22 - DNA, 20 bp, synthetic construct
  source 1..20: mol_type = other DNA
aaaaaaaaaaaaaaaaagag

SEQ ID NO: 23 - DNA, 20 bp, synthetic construct
  source 1..20: mol_type = other DNA
aaaaaaaaaaaaaaaaagat

SEQ ID NO: 24 - DNA, 20 bp, synthetic construct
  source 1..20: mol_type = other DNA
aaaaaaaaaaaaaaaaagca

SEQ ID NO: 25 - DNA, 20 bp, synthetic construct
  source 1..20: mol_type = other DNA
aaaaaaaaaaaaaaaaagcc

SEQ ID NO: 26 - DNA, 20 bp, synthetic construct
  source 1..20: mol_type = other DNA
aaaaaaaaaaaaaaaaagct

SEQ ID NO: 27 - DNA, 20 bp, synthetic construct
  source 1..20: mol_type = other DNA
aaaaaaaaaaaaaaaaagga

SEQ ID NO: 28 - DNA, 20 bp, synthetic construct
  source 1..20: mol_type = other DNA
aaaaaaaaaaaaaaaaaggc

SEQ ID NO: 29 - DNA, 20 bp, synthetic construct
  source 1..20: mol_type = other DNA
aaaaaaaaaaaaaaaaaggg

SEQ ID NO: 30 - DNA, 20 bp, synthetic construct
  source 1..20: mol_type = other DNA
aaaaaaaaaaaaaaaaaggt

SEQ ID NO: 31 - DNA, 20 bp, synthetic construct
  source 1..20: mol_type = other DNA
aaaaaaaaaaaaaaaaagta

SEQ ID NO: 32 - DNA, 20 bp, synthetic construct
  source 1..20: mol_type = other DNA
aaaaaaaaaaaaaaaaagtc

SEQ ID NO: 33 - DNA, 20 bp, synthetic construct
  source 1..20: mol_type = other DNA
aaaaaaaaaaaaaaaaagtg

SEQ ID NO: 34 - DNA, 20 bp, synthetic construct
  source 1..20: mol_type = other DNA
aaaaaaaaaaaaaaaaagtt

SEQ ID NO: 35 - DNA, 20 bp, synthetic construct
  source 1..20: mol_type = other DNA
aaaaaaaaaaaaaaaaatag

SEQ ID NO: 36 - DNA, 20 bp, synthetic construct
  source 1..20: mol_type = other DNA
aaaaaaaaaaaaaaaaatca

SEQ ID NO: 37 - DNA, 20 bp, synthetic construct
  source 1..20: mol_type = other DNA
aaaaaaaaaaaaaaaaattc

SEQ ID NO: 38 - DNA, 20 bp, synthetic construct
  source 1..20: mol_type = other DNA
aaaaaaaaaaaaaaaaattg

SEQ ID NO: 39 - DNA, 20 bp, synthetic construct
  source 1..20: mol_type = other DNA
aaaaaaaaaaaaaaaaattt

SEQ ID NO: 40 - DNA, 20 bp, synthetic construct
  source 1..20: mol_type = other DNA
aaaaaaaaaaaaaaaagaac

SEQ ID NO: 41 - DNA, 20 bp, synthetic construct
  source 1..20: mol_type = other DNA
aaaaaaaaaaaaaaaagaat

SEQ ID NO: 42 - DNA, 20 bp, synthetic construct
  source 1..20: mol_type = other DNA
aaaaaaaaaaaaaaaagaca

SEQ ID NO: 43 - DNA, 20 bp, synthetic construct
  source 1..20: mol_type = other DNA
aaaaaaaaaaaaaaaagagt

SEQ ID NO: 44 - DNA, 20 bp, synthetic construct
  source 1..20: mol_type = other DNA
aaaaaaaaaaaaaaaagata

SEQ ID NO: 45 - DNA, 20 bp, synthetic construct
  source 1..20: mol_type = other DNA
aaaaaaaaaaaaaaaagatt

SEQ ID NO: 46 - DNA, 20 bp, synthetic construct
  source 1..20: mol_type = other DNA
aaaaaaaaaaaaaaaagcaa

SEQ ID NO: 47 - DNA, 20 bp, synthetic construct
  source 1..20: mol_type = other DNA
aaaaaaaaaaaaaaaagcca

SEQ ID NO: 48 - DNA, 20 bp, synthetic construct
  source 1..20: mol_type = other DNA
aaaaaaaaaaaaaaaagtaa

SEQ ID NO: 49 - DNA, 20 bp, synthetic construct
  source 1..20: mol_type = other DNA
aaaaaaaaaaaaaaaattaa

SEQ ID NO: 50 - DNA, 20 bp, synthetic construct
  source 1..20: mol_type = other DNA
aaaaaaaaaaaaaaacaaaa

SEQ ID NO: 51 - DNA, 20 bp, synthetic construct
  source 1..20: mol_type = other DNA
aaaaaaaaaaaaaaagaaat

SEQ ID NO: 52 - DNA, 20 bp, synthetic construct
  source 1..20: mol_type = other DNA
aaaaaaaaaaaaaaagaata

SEQ ID NO: 53 - DNA, 20 bp, synthetic construct
  source 1..20: mol_type = other DNA
aaaaaaaaaaaaaaagaatt

SEQ ID NO: 54 - DNA, 20 bp, synthetic construct
  source 1..20: mol_type = other DNA
aaaaaaaaaaaaaacaaaaa

SEQ ID NO: 55 - DNA, 20 bp, synthetic construct
  source 1..20: mol_type = other DNA
aaaaaaaaaaaaacaaaaaa

SEQ ID NO: 56 - DNA, 20 bp, synthetic construct
  source 1..20: mol_type = other DNA
aaaaaaaaaaaacaaaaaaa

SEQ ID NO: 57 - DNA, 20 bp, synthetic construct
  source 1..20: mol_type = other DNA
aaaaaaacaaaacaaaacaa

SEQ ID NO: 58 - DNA, 20 bp, synthetic construct
  source 1..20: mol_type = other DNA
aaaaaaagaaaaaaaaaaaa

SEQ ID NO: 59 - DNA, 20 bp, synthetic construct
  source 1..20: mol_type = other DNA
aaaaaaataaataaataaaa

SEQ ID NO: 60 - DNA, 20 bp, synthetic construct
  source 1..20: mol_type = other DNA
aaaaaaatacaaaaattagc

SEQ ID NO: 61 - DNA, 20 bp, synthetic construct
  source 1..20: mol_type = other DNA
aaaaaacaaaaacaaaaaca

SEQ ID NO: 62 - DNA, 20 bp, synthetic construct
  source 1..20: mol_type = other DNA
aaaaaacaaaacaaaacaaa

SEQ ID NO: 63 - DNA, 20 bp, synthetic construct
  source 1..20: mol_type = other DNA
aaaaaacaaacaaacaaaaa

SEQ ID NO: 64 - DNA, 20 bp, synthetic construct
  source 1..20: mol_type = other DNA
aaaaaacaaacaaacaaaca

SEQ ID NO: 65 - DNA, 20 bp, synthetic construct
  source 1..20: mol_type = other DNA
aaaaaagaaaaaaaaaaaaa

SEQ ID NO: 66 - DNA, 20 bp, synthetic construct
  source 1..20: mol_type = other DNA
aaaaaataaaataaaataaa

SEQ ID NO: 67 - DNA, 20 bp, synthetic construct
  source 1..20: mol_type = other DNA
aaaaaataaataaataaata

SEQ ID NO: 68 - DNA, 20 bp, synthetic construct
  source 1..20: mol_type = other DNA
aaaaacaaaacaaaacaaaa

SEQ ID NO: 69 - DNA, 20 bp, synthetic construct
  source 1..20: mol_type = other DNA
aaaaacaaacaaacaaaaaa

SEQ ID NO: 70 - DNA, 20 bp, synthetic construct
  source 1..20: mol_type = other DNA
aaaaacaaacaaacaaacaa

SEQ ID NO: 71 - DNA, 20 bp, synthetic construct
  source 1..20: mol_type = other DNA
aaaaagaaaaaaaaaaaaaa

SEQ ID NO: 72 - DNA, 20 bp, synthetic construct
  source 1..20: mol_type = other DNA
aaaaataaaataaaataaaa

SEQ ID NO: 73 - DNA, 20 bp, synthetic construct
  source 1..20: mol_type = other DNA
aaaaataaataaataaataa

SEQ ID NO: 74 - DNA, 20 bp, synthetic construct
  source 1..20: mol_type = other DNA
aaaaataataataataataa

SEQ ID NO: 75 - DNA, 20 bp, synthetic construct
  source 1..20: mol_type = other DNA
aaaacaaaaacaaaaacaaa

SEQ ID NO: 76 - DNA, 20 bp, synthetic construct
  source 1..20: mol_type = other DNA
aaaacaaaacaaaacaaaaa

SEQ ID NO: 77 - DNA, 20 bp, synthetic construct
  source 1..20: mol_type = other DNA
aaaacaaacaaacaaaaaaa

SEQ ID NO: 78 - DNA, 20 bp, synthetic construct
  source 1..20: mol_type = other DNA
aaaacaaacaaacaaacaaa

SEQ ID NO: 79 - DNA, 20 bp, synthetic construct
  source 1..20: mol_type = other DNA
aaaacaacaacaacaacaac

SEQ ID NO: 80 - DNA, 20 bp, synthetic construct
  source 1..20: mol_type = other DNA
aaaataaataaataaataaa

SEQ ID NO: 81 - DNA, 20 bp, synthetic construct
  source 1..20: mol_type = other DNA
aaaataataataataataat

SEQ ID NO: 82 - DNA, 20 bp, synthetic construct
  source 1..20: mol_type = other DNA
aaacaaaacaaaacaaaaaa

SEQ ID NO: 83 - DNA, 20 bp, synthetic construct
  source 1..20: mol_type = other DNA
aaacaaacaaacaaaaaaaa

SEQ ID NO: 84 - DNA, 20 bp, synthetic construct
  source 1..20: mol_type = other DNA
aaacaaacaaacaaaaaaac

SEQ ID NO: 85 - DNA, 20 bp, synthetic construct
  source 1..20: mol_type = other DNA
aaacaaacaaacaaacaaaa

SEQ ID NO: 86 - DNA, 20 bp, synthetic construct
  source 1..20: mol_type = other DNA
aaacaacaacaacaacaaca

SEQ ID NO: 87 - DNA, 20 bp, synthetic construct
  source 1..20: mol_type = other DNA
aaacacacacacacacacac

SEQ ID NO: 88 - DNA, 20 bp, synthetic construct
  source 1..20: mol_type = other DNA
aaataataataataataata

SEQ ID NO: 89 - DNA, 20 bp, synthetic construct
  source 1..20: mol_type = other DNA
aaattttttttttttttttt

SEQ ID NO: 90 - DNA, 20 bp, synthetic construct
  source 1..20: mol_type = other DNA
aacaaaacaaaacaaaaaaa

SEQ ID NO: 91 - DNA, 20 bp, synthetic construct
  source 1..20: mol_type = other DNA
aacaaacaaacaaacaaaaa

SEQ ID NO: 92 - DNA, 20 bp, synthetic construct
  source 1..20: mol_type = other DNA
aacaacaacaacaacaaaaa

SEQ ID NO: 93 - DNA, 20 bp, synthetic construct
  source 1..20: mol_type = other DNA
aacacacacacacacacaca

SEQ ID NO: 94 - DNA, 20 bp, synthetic construct
  source 1..20: mol_type = other DNA
aacttttttttttttttttt

SEQ ID NO: 95 - DNA, 20 bp, synthetic construct
  source 1..20: mol_type = other DNA
aatacacacacacacacaca

SEQ ID NO: 96 - DNA, 20 bp, synthetic construct
  source 1..20: mol_type = other DNA
aatctttttttttttttttt

SEQ ID NO: 97 - DNA, 20 bp, synthetic construct
  source 1..20: mol_type = other DNA
aattcttttttttttttttt

SEQ ID NO: 98 - DNA, 20 bp, synthetic construct
  source 1..20: mol_type = other DNA
aatttttttttttttttttg

SEQ ID NO: 99 - DNA, 20 bp, synthetic construct
  source 1..20: mol_type = other DNA
aatttttttttttttttttt

SEQ ID NO: 100 - DNA, 20 bp, synthetic construct
  source 1..20: mol_type = other DNA
acaaaaaaaaaaaaaaaaaa

SEQ ID NO: 101 - DNA, 20 bp, synthetic construct
  source 1..20: mol_type = other DNA
acaaacaaacaaacaaaaaa

SEQ ID NO: 102 - DNA, 20 bp, synthetic construct
  source 1..20: mol_type = other DNA
acaacaacaacaacaacaaa

SEQ ID NO: 103 - DNA, 20 bp, synthetic construct
  source 1..20: mol_type = other DNA
acacacacacacacacacaa

SEQ ID NO: 104 - DNA, 20 bp, synthetic construct
  source 1..20: mol_type = other DNA
acacacacacacacacacag

SEQ ID NO: 105 - DNA, 20 bp, synthetic construct
  source 1..20: mol_type = other DNA
acacacacacacacacaccc

SEQ ID NO: 106 - DNA, 20 bp, synthetic construct
  source 1..20: mol_type = other DNA
accaaaaaaaaaaaaaaaaa

SEQ ID NO: 107 - DNA, 20 bp, synthetic construct
  source 1..20: mol_type = other DNA
accttttttttttttttttt

SEQ ID NO: 108 - DNA, 20 bp, synthetic construct
  source 1..20: mol_type = other DNA
actctttttttttttttttt

SEQ ID NO: 109 - RNA, 20 nt, synthetic construct
  source 1..20: mol_type = other RNA
actttttttttttttttttt

SEQ ID NO: 110 - DNA, 20 bp, synthetic construct
  source 1..20: mol_type = other DNA
agaaaaaaaaaaaaaaaaaa

SEQ ID NO: 111 - RNA, 20 nt, synthetic construct
  source 1..20: mol_type = other RNA
agcaaaaaaaaaaaaaaaaa

SEQ ID NO: 112 - DNA, 20 bp, synthetic construct
  source 1..20: mol_type = other DNA
agcttttttttttttttttt

SEQ ID NO: 113 - DNA, 20 bp, synthetic construct
  source 1..20: mol_type = other DNA
aggccaggcacggtggctca

SEQ ID NO: 114 - RNA, 20 nt, synthetic construct
  source 1..20: mol_type = other RNA
aggccaggcgtggtggctca

SEQ ID NO: 115 - DNA, 20 bp, synthetic construct
  source 1..20: mol_type = other DNA
aggccaggtgtggtggctca

SEQ ID NO: 116 - DNA, 20 bp, synthetic construct
  source 1..20: mol_type = other DNA
aggccgggcacggtggctca

SEQ ID NO: 117 - DNA, 20 bp, synthetic construct
  source 1..20: mol_type = other DNA
aggctgggcacagtggctca

SEQ ID NO: 118 - DNA, 20 bp, synthetic construct
  source 1..20: mol_type = other DNA
aggctgggcacggtggctca

SEQ ID NO: 119 - DNA, 20 bp, synthetic construct
  source 1..20: mol_type = other DNA
aggctgggcatggtggctca

SEQ ID NO: 120 - DNA, 20 bp, synthetic construct
  source 1..20: mol_type = other DNA
aggctgggtgcagtggctca

SEQ ID NO: 121 - DNA, 20 bp, synthetic construct
  source 1..20: mol_type = other DNA
aggctgggtgtggtggctca

SEQ ID NO: 122 - DNA, 20 bp, synthetic construct
  source 1..20: mol_type = other DNA
agtgtgtgtgtgtgtgtgtg

SEQ ID NO: 123 - DNA, 20 bp, synthetic construct
  source 1..20: mol_type = other DNA
agtttttttttttttttttt

SEQ ID NO: 124 - DNA, 20 bp, synthetic construct
  source 1..20: mol_type = other DNA
ataataataataataataaa

SEQ ID NO: 125 - DNA, 20 bp, synthetic construct
  source 1..20: mol_type = other DNA
atatatatatatatatataa

SEQ ID NO: 126 - DNA, 20 bp, synthetic construct
  source 1..20: mol_type = other DNA
atatggtttggctgtgtccc

SEQ ID NO: 127 - DNA, 20 bp, synthetic construct
  source 1..20: mol_type = other DNA
atcttttttttttttttttt

SEQ ID NO: 128 - DNA, 20 bp, synthetic construct
  source 1..20: mol_type = other DNA
atgagccaccatgcccagcc

SEQ ID NO: 129 - DNA, 20 bp, synthetic construct
  source 1..20: mol_type = other DNA
atgagccaccatgcctggcc

SEQ ID NO: 130 - DNA, 20 bp, synthetic construct
  source 1..20: mol_type = other DNA
atgagccaccgcgcccggcc

SEQ ID NO: 131 - DNA, 20 bp, synthetic construct
  source 1..20: mol_type = other DNA
atgagccaccgtgcctggcc

SEQ ID NO: 132 - DNA, 20 bp, synthetic construct
  source 1..20: mol_type = other DNA
atgagccactgcacctggcc

SEQ ID NO: 133 - DNA, 20 bp, synthetic construct
  source 1..20: mol_type = other DNA
atgagccactgtgcccagcc

SEQ ID NO: 134 - DNA, 20 bp, synthetic construct
  source 1..20: mol_type = other DNA
atgagccactgtgcctggcc

SEQ ID NO: 135 - DNA, 20 bp, synthetic construct
  source 1..20: mol_type = other DNA
attaaaaaaaaaaaaaaaaa

SEQ ID NO: 136 - DNA, 20 bp, synthetic construct
  source 1..20: mol_type = other DNA
attattattattattatttt

SEQ ID NO: 137 - DNA, 20 bp, synthetic construct
  source 1..20: mol_type = other DNA
attctttttttttttttttt

SEQ ID NO: 138 - DNA, 20 bp, synthetic construct
  source 1..20: mol_type = other DNA
atttcttttttttttttttt

SEQ ID NO: 139 - DNA, 20 bp, synthetic construct
  source 1..20: mol_type = other DNA
attttttttttttttgagac

SEQ ID NO: 140 - DNA, 20 bp, synthetic construct
  source 1..20: mol_type = other DNA
atttttttttttttttgaga

SEQ ID NO: 141 - DNA, 20 bp, synthetic construct
  source 1..20: mol_type = other DNA
attttttttttttttttgag

SEQ ID NO: 142 - DNA, 20 bp, synthetic construct
  source 1..20: mol_type = other DNA
atttttttttttttttttga

SEQ ID NO: 143 - DNA, 20 bp, synthetic construct
  source 1..20: mol_type = other DNA
attttttttttttttttttg

SEQ ID NO: 144 - DNA, 20 bp, synthetic construct
  source 1..20: mol_type = other DNA
attttttttttttttttttt

SEQ ID NO: 145 - DNA, 20 bp, synthetic construct
  source 1..20: mol_type = other DNA
caaaaaaaaaaaaaaaaaat

SEQ ID NO: 146 - DNA, 20 bp, synthetic construct
  source 1..20: mol_type = other DNA
caaaaaaaaaaaaaaaaatt

SEQ ID NO: 147 - DNA, 20 bp, synthetic construct
  source 1..20: mol_type = other DNA
caaacaaacaaacaaaaaaa

SEQ ID NO: 148 - DNA, 20 bp, synthetic construct
  source 1..20: mol_type = other DNA
caaacaaacaaacaaaaaac

SEQ ID NO: 149 - DNA, 20 bp, synthetic construct
  source 1..20: mol_type = other DNA
caacaacaacaacaacaaaa

SEQ ID NO: 150 - DNA, 20 bp, synthetic construct
  source 1..20: mol_type = other DNA
caattttttttttttttttt

SEQ ID NO: 151 - DNA, 20 bp, synthetic construct
  source 1..20: mol_type = other DNA
cacacacacacacacacaaa

SEQ ID NO: 152 - DNA, 20 bp, synthetic construct
  source 1..20: mol_type = other DNA
cacacacacacacacacacc

SEQ ID NO: 153 - DNA, 20 bp, synthetic construct
  source 1..20: mol_type = other DNA
cacacacacacacacacacg

SEQ ID NO: 154 - DNA, 20 bp, synthetic construct
  source 1..20: mol_type = other DNA
cacacacacacacacacact

SEQ ID NO: 155 - DNA, 20 bp, synthetic construct
  source 1..20: mol_type = other DNA
cacttttttttttttttttt

SEQ ID NO: 156 - DNA, 20 bp, synthetic construct
  source 1..20: mol_type = other DNA
catgagccaccacacctggc

SEQ ID NO: 157 - DNA, 20 bp, synthetic construct
  source 1..20: mol_type = other DNA
catgagccaccatgcccagc

SEQ ID NO: 158 - DNA, 20 bp, synthetic construct
  source 1..20: mol_type = other DNA
catgagccaccatgcctggc

SEQ ID NO: 159 - DNA, 20 bp, synthetic construct
  source 1..20: mol_type = other DNA
catgagccaccgcgcccggc

SEQ ID NO: 160 - DNA, 20 bp, synthetic construct
  source 1..20: mol_type = other DNA
catgagccaccgtgcctggc

SEQ ID NO: 161 - DNA, 20 bp, synthetic construct
  source 1..20: mol_type = other DNA
catgagccactgcacccagc

SEQ ID NO: 162 - DNA, 20 bp, synthetic construct
  source 1..20: mol_type = other DNA
catgagccactgcacctggc

SEQ ID NO: 163 - DNA, 20 bp, synthetic construct
  source 1..20: mol_type = other DNA
catgagccactgtgcccagc

SEQ ID NO: 164 - DNA, 20 bp, synthetic construct
  source 1..20: mol_type = other DNA
catgagccactgtgcctggc

SEQ ID NO: 165 - DNA, 20 bp, synthetic construct
  source 1..20: mol_type = other DNA
catttttttttttttttttt

SEQ ID NO: 166 - DNA, 20 bp, synthetic construct
  source 1..20: mol_type = other DNA
ccaaaaaaaaaaaaaaaaaa

SEQ ID NO: 167 - DNA, 20 bp, synthetic construct
  source 1..20: mol_type = other DNA
ccacacacacacacacacac

SEQ ID NO: 168 - DNA, 20 bp, synthetic construct
  source 1..20: mol_type = other DNA
ccaggcacagtggctcacac

SEQ ID NO: 169 - DNA, 20 bp, synthetic construct
  source 1..20: mol_type = other DNA
ccaggcacagtggctcacgc

SEQ ID NO: 170 - DNA, 20 bp, synthetic construct
  source 1..20: mol_type = other DNA
ccaggcacagtggctcatgc

SEQ ID NO: 171 - DNA, 20 bp, synthetic construct
  source 1..20: mol_type = other DNA
ccaggcacggtggctcacgc

SEQ ID NO: 172 - DNA, 20 bp, synthetic construct
  source 1..20: mol_type = other DNA
ccaggcacggtggctcatgc

SEQ ID NO: 173 - DNA, 20 bp, synthetic construct
  source 1..20: mol_type = other DNA
ccaggcatggtggctcacac

SEQ ID NO: 174 - DNA, 20 bp, synthetic construct
  source 1..20: mol_type = other DNA
ccaggcatggtggctcacgc

SEQ ID NO: 175 - DNA, 20 bp, synthetic construct
  source 1..20: mol_type = other DNA
ccaggcgcagtggctcacgc

SEQ ID NO: 176 - DNA, 20 bp, synthetic construct
  source 1..20: mol_type = other DNA
ccaggcgcggtggctcacgc

SEQ ID NO: 177 - DNA, 20 bp, synthetic construct
  source 1..20: mol_type = other DNA
ccaggcgtggtggctcacgc

SEQ ID NO: 178 - DNA, 20 bp, synthetic construct
  source 1..20: mol_type = other DNA
ccaggtgcagtggctcacac

SEQ ID NO: 179 - DNA, 20 bp, synthetic construct
  source 1..20: mol_type = other DNA
ccaggtgcagtggctcacgc

SEQ ID NO: 180 - DNA, 20 bp, synthetic construct
  source 1..20: mol_type = other DNA
ccaggtgcagtggctcatgc

SEQ ID NO: 181 - DNA, 20 bp, synthetic construct
  source 1..20: mol_type = other DNA
ccaggtgcggtggctcacgc

SEQ ID NO: 182 - DNA, 20 bp, synthetic construct
  source 1..20: mol_type = other DNA
cccttttttttttttttttt

SEQ ID NO: 183 - DNA, 20 bp, synthetic construct
  source 1..20: mol_type = other DNA
ccgggcacggtggctcacgc

SEQ ID NO: 184 - DNA, 20 bp, synthetic construct
  source 1..20: mol_type = other DNA
ccgggcgcagtggctcacgc

SEQ ID NO: 185 - DNA, 20 bp, synthetic construct
  source 1..20: mol_type = other DNA
ccgggcgcggtggctcacgc

SEQ ID NO: 186 - DNA, 20 bp, synthetic construct
  source 1..20: mol_type = other DNA
ccgggcgcggtggctcatgc

SEQ ID NO: 187 - DNA, 20 bp, synthetic construct
  source 1..20: mol_type = other DNA
ccgggcgtggtggctcacgc

SEQ ID NO: 188 - DNA, 20 bp, synthetic construct
  source 1..20: mol_type = other DNA
ccgggtgcggtggctcacgc

SEQ ID NO: 189 - DNA, 20 bp, synthetic construct
  source 1..20: mol_type = other DNA
cctttttttttttttttttt

SEQ ID NO: 190 - DNA, 20 bp, synthetic construct
  source 1..20: mol_type = other DNA
cgtgagccaccacgcccagc

SEQ ID NO: 191 - DNA, 20 bp, synthetic construct
  source 1..20: mol_type = other DNA
cgtgagccaccacgcccggc

SEQ ID NO: 192 - DNA, 20 bp, synthetic construct
  source 1..20: mol_type = other DNA
cgtgagccaccacgcctggc

SEQ ID NO: 193 - DNA, 20 bp, synthetic construct
  source 1..20: mol_type = other DNA
cgtgagccaccatgcccagc

SEQ ID NO: 194 - DNA, 20 bp, synthetic construct
  source 1..20: mol_type = other DNA
cgtgagccaccatgcctggc

SEQ ID NO: 195 - DNA, 20 bp, synthetic construct
  source 1..20: mol_type = other DNA
cgtgagccaccgcacccggc

SEQ ID NO: 196 - DNA, 20 bp, synthetic construct
  source 1..20: mol_type = other DNA
cgtgagccaccgcacctggc

SEQ ID NO: 197 - DNA, 20 bp, synthetic construct
  source 1..20: mol_type = other DNA
cgtgagccaccgcgcccagc

SEQ ID NO: 198 - DNA, 20 bp, synthetic construct
  source 1..20: mol_type = other DNA
cgtgagccaccgcgcccggc

SEQ ID NO: 199 - DNA, 20 bp, synthetic construct
  source 1..20: mol_type = other DNA
cgtgagccaccgcgcctggc

SEQ ID NO: 200 - DNA, 20 bp, synthetic construct
  source 1..20: mol_type = other DNA
cgtgagccaccgtgcccagc

SEQ ID NO: 201 - DNA, 20 bp, synthetic construct
  source 1..20: mol_type = other DNA
cgtgagccaccgtgcccggc

SEQ ID NO: 202 - DNA, 20 bp, synthetic construct
  source 1..20: mol_type = other DNA
cgtgagccaccgtgcctggc

SEQ ID NO: 203 - DNA, 20 bp, synthetic construct
  source 1..20: mol_type = other DNA
cgtgagccactgcacctggc

SEQ ID NO: 204 - DNA, 20 bp, synthetic construct
  source 1..20: mol_type = other DNA
cgtgagccactgcgcccagc

SEQ ID NO: 205 - DNA, 20 bp, synthetic construct
  source 1..20: mol_type = other DNA
cgtgagccactgcgcccggc

SEQ ID NO: 206 - DNA, 20 bp, synthetic construct
  source 1..20: mol_type = other DNA
cgtgagccactgcgcctggc

SEQ ID NO: 207 - DNA, 20 bp, synthetic construct
  source 1..20: mol_type = other DNA
cgtgagccactgtgcccagc

SEQ ID NO: 208 - DNA, 20 bp, synthetic construct
  source 1..20: mol_type = other DNA
cgtgagccactgtgcctggc

SEQ ID NO: 209 - DNA, 20 bp, synthetic construct
  source 1..20: mol_type = other DNA
cgtgtgtgtgtgtgtgtgtg

SEQ ID NO: 210 - DNA, 20 bp, synthetic construct
  source 1..20: mol_type = other DNA
ctattttttttttttttttt

SEQ ID NO: 211 - DNA, 20 bp, synthetic construct
  source 1..20: mol_type = other DNA
ctcaaaaaaaaaaaaaaaat

SEQ ID NO: 212 - DNA, 20 bp, synthetic construct
  source 1..20: mol_type = other DNA
ctcttttttttttttttttt

SEQ ID NO: 213 - DNA, 20 bp, synthetic construct
  source 1..20: mol_type = other DNA
ctgggcacagtggctcacgc

SEQ ID NO: 214 - DNA, 20 bp, synthetic construct
  source 1..20: mol_type = other DNA
ctgggcacagtggctcatgc

SEQ ID NO: 215 - DNA, 20 bp, synthetic construct
  source 1..20: mol_type = other DNA
ctgggcacggtggctcacgc

SEQ ID NO: 216 - DNA, 20 bp, synthetic construct
  source 1..20: mol_type = other DNA
ctgggcacggtggctcatgc

SEQ ID NO: 217 - DNA, 20 bp, synthetic construct
  source 1..20: mol_type = other DNA
ctgggcatggtggctcacac

SEQ ID NO: 218 - DNA, 20 bp, synthetic construct
  source 1..20: mol_type = other DNA
ctgggcatggtggctcacgc

SEQ ID NO: 219 - DNA, 20 bp, synthetic construct
  source 1..20: mol_type = other DNA
ctgggcatggtggctcatgc

SEQ ID NO: 220 - DNA, 20 bp, synthetic construct
  source 1..20: mol_type = other DNA
ctgggcgcagtggctcacgc

SEQ ID NO: 221 - DNA, 20 bp, synthetic construct
  source 1..20: mol_type = other DNA
ctgggcgcggtggctcacgc

SEQ ID NO: 222 - DNA, 20 bp, synthetic construct
  source 1..20: mol_type = other DNA
ctgggcgtggtggctcacgc

SEQ ID NO: 223 - DNA, 20 bp, synthetic construct
  source 1..20: mol_type = other DNA
ctgggtgcagtggctcatgc

SEQ ID NO: 224 - DNA, 20 bp, synthetic construct
  source 1..20: mol_type = other DNA
ctgggtgtggtggctcatgc

SEQ ID NO: 225 - DNA, 20 bp, synthetic construct
  source 1..20: mol_type = other DNA
ctgtgtgtgtgtgtgtgtgt

SEQ ID NO: 226 - DNA, 20 bp, synthetic construct
  source 1..20: mol_type = other DNA
ctttttttttttttttttta

SEQ ID NO: 227 - DNA, 20 bp, synthetic construct
  source 1..20: mol_type = other DNA
cttttttttttttttttttt

SEQ ID NO: 228 - DNA, 20 bp, synthetic construct
  source 1..20: mol_type = other DNA
gaaaaaaaaaaaaaaaaaaa

SEQ ID NO: 229 - DNA, 20 bp, synthetic construct
  source 1..20: mol_type = other DNA
gaattttttttttttttttt

SEQ ID NO: 230 - DNA, 20 bp, synthetic construct
  source 1..20: mol_type = other DNA
gacacagccaaaccatatca

SEQ ID NO: 231 - DNA, 20 bp, synthetic construct
  source 1..20: mol_type = other DNA
gacttttttttttttttttt

SEQ ID NO: 232 - DNA, 20 bp, synthetic construct
  source 1..20: mol_type = other DNA
gatatggtttggctgtgtcc

SEQ ID NO: 233 - DNA, 20 bp, synthetic construct
  source 1..20: mol_type = other DNA
gatttttttttttttttttt

SEQ ID NO: 234 - DNA, 20 bp, synthetic construct
  source 1..20: mol_type = other DNA
gcaaaaaaaaaaaaaaaaaa

SEQ ID NO: 235 - DNA, 20 bp, synthetic construct
  source 1..20: mol_type = other DNA
gcatgagccaccacacccag

SEQ ID NO: 236 - DNA, 20 bp, synthetic construct
  source 1..20: mol_type = other DNA
gcatgagccaccatgcccag

SEQ ID NO: 237 - DNA, 20 bp, synthetic construct
  source 1..20: mol_type = other DNA
gcatgagccaccgcgcccgg

SEQ ID NO: 238 - DNA, 20 bp, synthetic construct
  source 1..20: mol_type = other DNA
gcatgagccaccgtgcccag

SEQ ID NO: 239 - DNA, 20 bp, synthetic construct
  source 1..20: mol_type = other DNA
gcatgagccaccgtgcctgg

SEQ ID NO: 240 - DNA, 20 bp, synthetic construct
  source 1..20: mol_type = other DNA
gcatgagccactgcacccag

SEQ ID NO: 241 - DNA, 20 bp, synthetic construct
  source 1..20: mol_type = other DNA
gcatgagccactgcacctgg

SEQ ID NO: 242 - DNA, 20 bp, synthetic construct
  source 1..20: mol_type = other DNA
gcatgagccactgtgcccag

SEQ ID NO: 243 - DNA, 20 bp, synthetic construct
  source 1..20: mol_type = other DNA
gcatgagccactgtgcctgg

SEQ ID NO: 244 - DNA, 20 bp, synthetic construct
  source 1..20: mol_type = other DNA
gccaggcacagtggctcaca

SEQ ID NO: 245 - DNA, 20 bp, synthetic construct
  source 1..20: mol_type = other DNA
gccaggcacagtggctcacg

SEQ ID NO: 246 - DNA, 20 bp, synthetic construct
  source 1..20: mol_type = other DNA
gccaggcacagtggctcatg

SEQ ID NO: 247 - DNA, 20 bp, synthetic construct
  source 1..20: mol_type = other DNA
gccaggcacggtggctcacg

SEQ ID NO: 248 - DNA, 20 bp, synthetic construct
  source 1..20: mol_type = other DNA
gccaggcacggtggctcatg

SEQ ID NO: 249 - DNA, 20 bp, synthetic construct
  source 1..20: mol_type = other DNA
gccaggcatggtggctcaca

SEQ ID NO: 250 - DNA, 20 bp, synthetic construct
  source 1..20: mol_type = other DNA
gccaggcatggtggctcacg

SEQ ID NO: 251 - DNA, 20 bp, synthetic construct
  source 1..20: mol_type = other DNA
gccaggcatggtggctcatg

SEQ ID NO: 252 - DNA, 20 bp, synthetic construct
  source 1..20: mol_type = other DNA
gccaggcgcagtggctcacg

SEQ ID NO: 253 - DNA, 20 bp, synthetic construct
  source 1..20: mol_type = other DNA
gccaggcgcggtggctcacg

SEQ ID NO: 254 - DNA, 20 bp, synthetic construct
  source 1..20: mol_type = other DNA
gccaggcgtggtggctcacg

SEQ ID NO: 255 - DNA, 20 bp, synthetic construct
  source 1..20: mol_type = other DNA
gccaggtgcagtggctcaca

SEQ ID NO: 256 - DNA, 20 bp, synthetic construct
  source 1..20: mol_type = other DNA
gccaggtgcagtggctcacg

SEQ ID NO: 257 - DNA, 20 bp, synthetic construct
  source 1..20: mol_type = other DNA
gccaggtgcagtggctcatg

SEQ ID NO: 258 - DNA, 20 bp, synthetic construct
  source 1..20: mol_type = other DNA
gccaggtgcggtggctcacg

SEQ ID NO: 259 - DNA, 20 bp, synthetic construct
  source 1..20: mol_type = other DNA
gccaggtgtggtggctcatg

SEQ ID NO: 260 - DNA, 20 bp, synthetic construct
  source 1..20: mol_type = other DNA
gccgggcacggtggctcacg

SEQ ID NO: 261 - DNA, 20 bp, synthetic construct
  source 1..20: mol_type = other DNA
gccgggcgcagtggctcacg

SEQ ID NO: 262 - DNA, 20 bp, synthetic construct
  source 1..20: mol_type = other DNA
gccgggcgcggtggctcacg

SEQ ID NO: 263 - DNA, 20 bp, synthetic construct
  source 1..20: mol_type = other DNA
gccgggcgcggtggctcatg

SEQ ID NO: 264 - DNA, 20 bp, synthetic construct
  source 1..20: mol_type = other DNA
gccgggcgtggtggctcacg

SEQ ID NO: 265 - DNA, 20 bp, synthetic construct
  source 1..20: mol_type = other DNA
gccgggtgcggtggctcacg

SEQ ID NO: 266 - DNA, 20 bp, synthetic construct
  source 1..20: mol_type = other DNA
gccttttttttttttttttt

SEQ ID NO: 267 - DNA, 20 bp, synthetic construct
  source 1..20: mol_type = other DNA
gcgtgagccaccacgcccag

SEQ ID NO: 268 - DNA, 20 bp, synthetic construct
  source 1..20: mol_type = other DNA
gcgtgagccaccacgcccgg

SEQ ID NO: 269 - DNA, 20 bp, synthetic construct
  source 1..20: mol_type = other DNA
gcgtgagccaccacgcctgg

SEQ ID NO: 270 - DNA, 20 bp, synthetic construct
  source 1..20: mol_type = other DNA
gcgtgagccaccatgcccag

SEQ ID NO: 271 - DNA, 20 bp, synthetic construct
  source 1..20: mol_type = other DNA
gcgtgagccaccatgcctgg

SEQ ID NO: 272 - DNA, 20 bp, synthetic construct
  source 1..20: mol_type = other DNA
gcgtgagccaccgcacccgg

SEQ ID NO: 273 - DNA, 20 bp, synthetic construct
  source 1..20: mol_type = other DNA
gcgtgagccaccgcacctgg

SEQ ID NO: 274 - DNA, 20 bp, synthetic construct
  source 1..20: mol_type = other DNA
gcgtgagccaccgcgcccag

SEQ ID NO: 275 - DNA, 20 bp, synthetic construct
  source 1..20: mol_type = other DNA
gcgtgagccaccgcgcccgg

SEQ ID NO: 276 - DNA, 20 bp, synthetic construct
  source 1..20: mol_type = other DNA
gcgtgagccaccgcgcctgg

SEQ ID NO: 277 - DNA, 20 bp, synthetic construct
  source 1..20: mol_type = other DNA
gcgtgagccaccgtgcccag

SEQ ID NO: 278 - DNA, 20 bp, synthetic construct
  source 1..20: mol_type = other DNA
gcgtgagccaccgtgcccgg

SEQ ID NO: 279 - DNA, 20 bp, synthetic construct
  source 1..20: mol_type = other DNA
gcgtgagccaccgtgcctgg

SEQ ID NO: 280 - DNA, 20 bp, synthetic construct
  source 1..20: mol_type = other DNA
gcgtgagccactgcacctgg

SEQ ID NO: 281 - DNA, 20 bp, synthetic construct
  source 1..20: mol_type = other DNA
gcgtgagccactgcgcccag

SEQ ID NO: 282 - DNA, 20 bp, synthetic construct
  source 1..20: mol_type = other DNA
gcgtgagccactgcgcccgg

SEQ ID NO: 283 - DNA, 20 bp, synthetic construct
  source 1..20: mol_type = other DNA
gcgtgagccactgcgcctgg

SEQ ID NO: 284 - DNA, 20 bp, synthetic construct
  source 1..20: mol_type = other DNA
gcgtgagccactgtgcccag

SEQ ID NO: 285 - DNA, 20 bp, synthetic construct
  source 1..20: mol_type = other DNA
gcgtgagccactgtgcctgg

SEQ ID NO: 286 - DNA, 20 bp, synthetic construct
  source 1..20: mol_type = other DNA
gctaatttttgtattttttt

SEQ ID NO: 287 - DNA, 20 bp, synthetic construct
  source 1..20: mol_type = other DNA
gctgggcacagtggctcacg

SEQ ID NO: 288 - DNA, 20 bp, synthetic construct
  source 1..20: mol_type = other DNA
gctgggcacagtggctcatg

SEQ ID NO: 289 - DNA, 20 bp, synthetic construct
  source 1..20: mol_type = other DNA
gctgggcacggtggctcacg

SEQ ID NO: 290 - DNA, 20 bp, synthetic construct
  source 1..20: mol_type = other DNA
gctgggcatggtggctcaca

SEQ ID NO: 291 - DNA, 20 bp, synthetic construct
  source 1..20: mol_type = other DNA
gctgggcatggtggctcacg

SEQ ID NO: 292 - DNA, 20 bp, synthetic construct
  source 1..20: mol_type = other DNA
gctgggcatggtggctcatg

SEQ ID NO: 293 - DNA, 20 bp, synthetic construct
  source 1..20: mol_type = other DNA
gctgggcgcagtggctcacg

SEQ ID NO: 294 - DNA, 20 bp, synthetic construct
  source 1..20: mol_type = other DNA
gctgggcgcggtggctcacg

SEQ ID NO: 295 - DNA, 20 bp, synthetic construct
  source 1..20: mol_type = other DNA
gctgggcgtggtggctcacg

SEQ ID NO: 296 - DNA, 20 bp, synthetic construct
  source 1..20: mol_type = other DNA
gctgggtgcagtggctcatg

SEQ ID NO: 297 - DNA, 20 bp, synthetic construct
  source 1..20: mol_type = other DNA
gctttttttttttttttttt

SEQ ID NO: 298 - DNA, 20 bp, synthetic construct
  source 1..20: mol_type = other DNA
ggaaaaaaaaaaaaaaaaaa

SEQ ID NO: 299 - DNA, 20 bp, synthetic construct
  source 1..20: mol_type = other DNA
ggacacagccaaaccatatc

SEQ ID NO: 300 - DNA, 20 bp, synthetic construct
  source 1..20: mol_type = other DNA
ggccaggcacagtggctcac

SEQ ID NO: 301 - DNA, 20 bp, synthetic construct
  source 1..20: mol_type = other DNA
ggccaggcacagtggctcat

SEQ ID NO: 302 - DNA, 20 bp, synthetic construct
  source 1..20: mol_type = other DNA
ggccaggcacggtggctcac

SEQ ID NO: 303 - DNA, 20 bp, synthetic construct
  source 1..20: mol_type = other DNA
ggccaggcacggtggctcat

SEQ ID NO: 304 - DNA, 20 bp, synthetic construct
  source 1..20: mol_type = other DNA
ggccaggcatggtggctcac

SEQ ID NO: 305 - DNA, 20 bp, synthetic construct
  source 1..20: mol_type = other DNA
ggccaggcatggtggctcat

SEQ ID NO: 306 - DNA, 20 bp, synthetic construct
  source 1..20: mol_type = other DNA
ggccaggcgcagtggctcac

SEQ ID NO: 307 - DNA, 20 bp, synthetic construct
  source 1..20: mol_type = other DNA
ggccaggcgcggtggctcac

SEQ ID NO: 308 - DNA, 20 bp, synthetic construct
  source 1..20: mol_type = other DNA
ggccaggcgtggtggctcac

SEQ ID NO: 309 - DNA, 20 bp, synthetic construct
  source 1..20: mol_type = other DNA
ggccaggtgcagtggctcac

SEQ ID NO: 310 - DNA, 20 bp, synthetic construct
  source 1..20: mol_type = other DNA
ggccaggtgcagtggctcat

SEQ ID NO: 311 - DNA, 20 bp, synthetic construct
  source 1..20: mol_type = other DNA
ggccaggtgcggtggctcac

SEQ ID NO: 312 - DNA, 20 bp, synthetic construct
  source 1..20: mol_type = other DNA
ggccaggtgtggtggctcac

SEQ ID NO: 313 - DNA, 20 bp, synthetic construct
  source 1..20: mol_type = other DNA
ggccgggcacagtggctcac

SEQ ID NO: 314 - DNA, 20 bp, synthetic construct
  source 1..20: mol_type = other DNA
ggccgggcacggtggctcac

SEQ ID NO: 315 - DNA, 20 bp, synthetic construct
  source 1..20: mol_type = other DNA
ggccgggcatggtggctcac

SEQ ID NO: 316 - DNA, 20 bp, synthetic construct
  source 1..20: mol_type = other DNA
ggccgggcgcagtggctcac

SEQ ID NO: 317 - DNA, 20 bp, synthetic construct
  source 1..20: mol_type = other DNA
ggccgggcgcggtggctcac

SEQ ID NO: 318 - DNA, 20 bp, synthetic construct
  source 1..20: mol_type = other DNA
ggccgggcgcggtggctcat

SEQ ID NO: 319 - DNA, 20 bp, synthetic construct
  source 1..20: mol_type = other DNA
ggccgggcgtggtggctcac

SEQ ID NO: 320 - DNA, 20 bp, synthetic construct
  source 1..20: mol_type = other DNA
ggccgggtgcagtggctcac

SEQ ID NO: 321 - DNA, 20 bp, synthetic construct
  source 1..20: mol_type = other DNA
ggccgggtgcggtggctcac

SEQ ID NO: 322 - DNA, 20 bp, synthetic construct
  source 1..20: mol_type = other DNA
ggccgggtgtggtggctcac

SEQ ID NO: 323 - DNA, 20 bp, synthetic construct
  source 1..20: mol_type = other DNA
ggctgggcacagtggctcac

SEQ ID NO: 324 - DNA, 20 bp, synthetic construct
  source 1..20: mol_type = other DNA
ggctgggcacagtggctcat

SEQ ID NO: 325 - DNA, 20 bp, synthetic construct
  source 1..20: mol_type = other DNA
ggctgggcacggtggctcac

SEQ ID NO: 326 - DNA, 20 bp, synthetic construct
  source 1..20: mol_type = other DNA
ggctgggcatggtggctcac

SEQ ID NO: 327 - DNA, 20 bp, synthetic construct
  source 1..20: mol_type = other DNA
ggctgggcatggtggctcat

SEQ ID NO: 328 - DNA, 20 bp, synthetic construct
  source 1..20: mol_type = other DNA
ggctgggcgcagtggctcac

SEQ ID NO: 329 - DNA, 20 bp, synthetic construct
  source 1..20: mol_type = other DNA
ggctgggcgcggtggctcac

SEQ ID NO: 330 - DNA, 20 bp, synthetic construct
  source 1..20: mol_type = other DNA
ggctgggcgtggtggctcac

SEQ ID NO: 331 - DNA, 20 bp, synthetic construct
  source 1..20: mol_type = other DNA
ggctgggtgcagtggctcac

SEQ ID NO: 332 - DNA, 20 bp, synthetic construct
  source 1..20: mol_type = other DNA
ggctgggtgcggtggctcac

SEQ ID NO: 333 - DNA, 20 bp, synthetic construct
  source 1..20: mol_type = other DNA
ggctgggtgtggtggctcac

SEQ ID NO: 334 - DNA, 20 bp, synthetic construct
  source 1..20: mol_type = other DNA
ggcttttttttttttttttt

SEQ ID NO: 335 - DNA, 20 bp, synthetic construct
  source 1..20: mol_type = other DNA
gggacacagccaaaccatat

SEQ ID NO: 336 - DNA, 20 bp, synthetic construct
  source 1..20: mol_type = other DNA
gggccgggcgcggtggctca

SEQ ID NO: 337 - DNA, 20 bp, synthetic construct
  source 1..20: mol_type = other DNA
gggtgtgtgtgtgtgtgtgt

SEQ ID NO: 338 - DNA, 20 bp, synthetic construct
  source 1..20: mol_type = other DNA
ggtgtgtgtgtgtgtgtgtg

SEQ ID NO: 339 - DNA, 20 bp, synthetic construct
  source 1..20: mol_type = other DNA
ggtttttttttttttttttt

SEQ ID NO: 340 - DNA, 20 bp, synthetic construct
  source 1..20: mol_type = other DNA
gtaaaaaaaaaaaaaaaaaa

SEQ ID NO: 341 - DNA, 20 bp, synthetic construct
  source 1..20: mol_type = other DNA
gtctcaaaaaaaaaaaaaat

SEQ ID NO: 342 - DNA, 20 bp, synthetic construct
  source 1..20: mol_type = other DNA
gtcttttttttttttttttt

SEQ ID NO: 343 - DNA, 20 bp, synthetic construct
  source 1..20: mol_type = other DNA
gtgagccaccacacccagcc

SEQ ID NO: 344 - DNA, 20 bp, synthetic construct
  source 1..20: mol_type = other DNA
gtgagccaccacacccggcc

SEQ ID NO: 345 - DNA, 20 bp, synthetic construct
  source 1..20: mol_type = other DNA
gtgagccaccacacctggcc

SEQ ID NO: 346 - DNA, 20 bp, synthetic construct
  source 1..20: mol_type = other DNA
gtgagccaccacgcccagcc

SEQ ID NO: 347 - DNA, 20 bp, synthetic construct
  source 1..20: mol_type = other DNA
gtgagccaccacgcccggcc

SEQ ID NO: 348 - DNA, 20 bp, synthetic construct
  source 1..20: mol_type = other DNA
gtgagccaccacgcctggcc

SEQ ID NO: 349 - DNA, 20 bp, synthetic construct
  source 1..20: mol_type = other DNA
gtgagccaccatgcccagcc

SEQ ID NO: 350 - DNA, 20 bp, synthetic construct
  source 1..20: mol_type = other DNA
gtgagccaccatgcccggcc

SEQ ID NO: 351 - DNA, 20 bp, synthetic construct
  source 1..20: mol_type = other DNA
gtgagccaccatgcctggcc

SEQ ID NO: 352 - DNA, 20 bp, synthetic construct
  source 1..20: mol_type = other DNA
gtgagccaccgcacccagcc

SEQ ID NO: 353 - RNA, 20 nt, synthetic construct
  source 1..20: mol_type = other RNA
gtgagccaccgcacccggcc

SEQ ID NO: 354 - DNA, 20 bp, synthetic construct
  source 1..20: mol_type = other DNA
gtgagccaccgcacctggcc

SEQ ID NO: 355 - RNA, 20 nt, synthetic construct
  source 1..20: mol_type = other RNA
gtgagccaccgcgcccagcc

SEQ ID NO: 356 - DNA, 20 bp, synthetic construct
  source 1..20: mol_type = other DNA
gtgagccaccgcgcccggcc

SEQ ID NO: 357 - DNA, 20 bp, synthetic construct
  source 1..20: mol_type = other DNA
gtgagccaccgcgcctggcc

SEQ ID NO: 358 - DNA, 20 bp, synthetic construct
  source 1..20: mol_type = other DNA
gtgagccaccgtgcccagcc

SEQ ID NO: 359 - DNA, 20 bp, synthetic construct
  source 1..20: mol_type = other DNA
gtgagccaccgtgcccggcc

SEQ ID NO: 360 - DNA, 20 bp, synthetic construct
  source 1..20: mol_type = other DNA
gtgagccaccgtgcctggcc

SEQ ID NO: 361 - DNA, 20 bp, synthetic construct
  source 1..20: mol_type = other DNA
gtgagccactgcacccagcc

SEQ ID NO: 362 - DNA, 20 bp, synthetic construct
  source 1..20: mol_type = other DNA
gtgagccactgcacccggcc

SEQ ID NO: 363 - DNA, 20 bp, synthetic construct
  source 1..20: mol_type = other DNA
gtgagccactgcacctggcc

SEQ ID NO: 364 - DNA, 20 bp, synthetic construct
  source 1..20: mol_type = other DNA
gtgagccactgcgcccagcc

SEQ ID NO: 365 - DNA, 20 bp, synthetic construct
  source 1..20: mol_type = other DNA
gtgagccactgcgcccggcc

SEQ ID NO: 366 - DNA, 20 bp, synthetic construct
  source 1..20: mol_type = other DNA
gtgagccactgcgcctggcc

SEQ ID NO: 367 - DNA, 20 bp, synthetic construct
  source 1..20: mol_type = other DNA
gtgagccactgtgcccagcc

SEQ ID NO: 368 - DNA, 20 bp, synthetic construct
  source 1..20: mol_type = other DNA
gtgagccactgtgcccggcc

SEQ ID NO: 369 - DNA, 20 bp, synthetic construct
  source 1..20: mol_type = other DNA
gtgagccactgtgcctggcc

SEQ ID NO: 370 - DNA, 20 bp, synthetic construct
  source 1..20: mol_type = other DNA
gtgtgagccaccatgcccag

SEQ ID NO: 371 - DNA, 20 bp, synthetic construct
  source 1..20: mol_type = other DNA
gtgtgagccaccatgcctgg

SEQ ID NO: 372 - DNA, 20 bp, synthetic construct
  source 1..20: mol_type = other DNA
gtgtgagccactgcacctgg

SEQ ID NO: 373 - DNA, 20 bp, synthetic construct
  source 1..20: mol_type = other DNA
gtgtgagccactgtgcctgg

SEQ ID NO: 374 - DNA, 20 bp, synthetic construct
  source 1..20: mol_type = other DNA
gtgtgtgtgtgtgtgtgtgg

SEQ ID NO: 375 - DNA, 20 bp, synthetic construct
  source 1..20: mol_type = other DNA
gtgtgtgtgtgtgtgtgttt

SEQ ID NO: 376 - DNA, 20 bp, synthetic construct
  source 1..20: mol_type = other DNA
gttctttttttttttttttt

SEQ ID NO: 377 - DNA, 20 bp, synthetic construct
  source 1..20: mol_type = other DNA
gttgttgttgttgttgtttt

SEQ ID NO: 378 - DNA, 20 bp, synthetic construct
  source 1..20: mol_type = other DNA
gttttttgtttgtttgtttg

SEQ ID NO: 379 - DNA, 20 bp, synthetic construct
  source 1..20: mol_type = other DNA
gtttttttgtttgtttgttt

SEQ ID NO: 380 - DNA, 20 bp, synthetic construct
  source 1..20: mol_type = other DNA
gttttttttttttttttttt

SEQ ID NO: 381 - DNA, 20 bp, synthetic construct
  source 1..20: mol_type = other DNA
taaaaaaaaaaaaaaaaaaa

SEQ ID NO: 382 - DNA, 20 bp, synthetic construct
  source 1..20: mol_type = other DNA
taaaaaaaaaaaaaaaaaag

SEQ ID NO: 383 - DNA, 20 bp, synthetic construct
  source 1..20: mol_type = other DNA
tacttttttttttttttttt

SEQ ID NO: 384 - DNA, 20 bp, synthetic construct
  source 1..20: mol_type = other DNA
tatatatatatatatataaa

SEQ ID NO: 385 - DNA, 20 bp, synthetic construct
  source 1..20: mol_type = other DNA
tatctttttttttttttttt

SEQ ID NO: 386 - DNA, 20 bp, synthetic construct
  source 1..20: mol_type = other DNA
tattattattattattattt

SEQ ID NO: 387 - DNA, 20 bp, synthetic construct
  source 1..20: mol_type = other DNA
tattcttttttttttttttt

SEQ ID NO: 388 - DNA, 20 bp, synthetic construct
  source 1..20: mol_type = other DNA
tatttatttatttatttttt

SEQ ID NO: 389 - DNA, 20 bp, synthetic construct
  source 1..20: mol_type = other DNA
tcaaaaaaaaaaaaaaaaat

SEQ ID NO: 390 - DNA, 20 bp, synthetic construct
  source 1..20: mol_type = other DNA
tccttttttttttttttttt

SEQ ID NO: 391 - DNA, 20 bp, synthetic construct
  source 1..20: mol_type = other DNA
tctcaaaaaaaaaaaaaaat

SEQ ID NO: 392 - DNA, 20 bp, synthetic construct
  source 1..20: mol_type = other DNA
tgagccaccacacccagcct

SEQ ID NO: 393 - DNA, 20 bp, synthetic construct
  source 1..20: mol_type = other DNA
tgagccaccacacctggcct

SEQ ID NO: 394 - DNA, 20 bp, synthetic construct
  source 1..20: mol_type = other DNA
tgagccaccacgcctggcct

SEQ ID NO: 395 - DNA, 20 bp, synthetic construct
  source 1..20: mol_type = other DNA
tgagccaccatgcccagcca

SEQ ID NO: 396 - DNA, 20 bp, synthetic construct
  source 1..20: mol_type = other DNA
tgagccaccatgcccagcct

SEQ ID NO: 397 - DNA, 20 bp, synthetic construct
  source 1..20: mol_type = other DNA
tgagccaccatgcctggcca

SEQ ID NO: 398 - DNA, 20 bp, synthetic construct
  source 1..20: mol_type = other DNA
tgagccaccgcgcccggcca

SEQ ID NO: 399 - DNA, 20 bp, synthetic construct
  source 1..20: mol_type = other DNA
tgagccaccgcgcccggccc

SEQ ID NO: 400 - DNA, 20 bp, synthetic construct
  source 1..20: mol_type = other DNA
tgagccaccgtgcccagcct

SEQ ID NO: 401 - DNA, 20 bp, synthetic construct
  source 1..20: mol_type = other DNA
tgagccaccgtgcccggcct

SEQ ID NO: 402 - DNA, 20 bp, synthetic construct
  source 1..20: mol_type = other DNA
tgagccaccgtgcctggcct

SEQ ID NO: 403 - DNA, 20 bp, synthetic construct
  source 1..20: mol_type = other DNA
tgagccactgcacccagcct

SEQ ID NO: 404 - DNA, 20 bp, synthetic construct
  source 1..20: mol_type = other DNA
tgagccactgtgcccagcct

SEQ ID NO: 405 - DNA, 20 bp, synthetic construct
  source 1..20: mol_type = other DNA
tgagccactgtgcctggcca

SEQ ID NO: 406 - DNA, 20 bp, synthetic construct
  source 1..20: mol_type = other DNA
tgatatggtttggctgtgtc

SEQ ID NO: 407 - DNA, 20 bp, synthetic construct
  source 1..20: mol_type = other DNA
tgattttttttttttttttt

SEQ ID NO: 408 - DNA, 20 bp, synthetic construct
  source 1..20: mol_type = other DNA
tgcttttttttttttttttt

SEQ ID NO: 409 - DNA, 20 bp, synthetic construct
  source 1..20: mol_type = other DNA
tggccaggcacagtggctca

SEQ ID NO: 410 - DNA, 20 bp, synthetic construct
  source 1..20: mol_type = other DNA
tggccaggcatggtggctca

SEQ ID NO: 411 - DNA, 20 bp, synthetic construct
  source 1..20: mol_type = other DNA
tggccgggcgcggtggctca

SEQ ID NO: 412 - DNA, 20 bp, synthetic construct
  source 1..20: mol_type = other DNA
tggctgggcatggtggctca

SEQ ID NO: 413 - DNA, 20 bp, synthetic construct
  source 1..20: mol_type = other DNA
tggctttttttttttttttt

SEQ ID NO: 414 - DNA, 20 bp, synthetic construct
  source 1..20: mol_type = other DNA
tgtctttttttttttttttt

SEQ ID NO: 415 - DNA, 20 bp, synthetic construct
  source 1..20: mol_type = other DNA
tgtgagccaccatgcccagc

SEQ ID NO: 416 - DNA, 20 bp, synthetic construct
  source 1..20: mol_type = other DNA
tgtgagccaccatgcctggc

SEQ ID NO: 417 - DNA, 20 bp, synthetic construct
  source 1..20: mol_type = other DNA
tgtgagccactgcacctggc

SEQ ID NO: 418 - DNA, 20 bp, synthetic construct
  source 1..20: mol_type = other DNA
tgtgagccactgtgcctggc

SEQ ID NO: 419 - DNA, 20 bp, synthetic construct
  source 1..20: mol_type = other DNA
tgtgtgtgtgtgtgtgtatt

SEQ ID NO: 420 - DNA, 20 bp, synthetic construct
  source 1..20: mol_type = other DNA
tgtgtgtgtgtgtgtgtgtt

SEQ ID NO: 421 - DNA, 20 bp, synthetic construct
  source 1..20: mol_type = other DNA
tgttgttgttgttgttgttt

SEQ ID NO: 422 - DNA, 20 bp, synthetic construct
  source 1..20: mol_type = other DNA
tgtttgtttgtttgtttttt

SEQ ID NO: 423 - DNA, 20 bp, synthetic construct
  source 1..20: mol_type = other DNA
tgtttttgtttttgtttttt

SEQ ID NO: 424 - DNA, 20 bp, synthetic construct
  source 1..20: mol_type = other DNA
tgtttttttttttttttttt

SEQ ID NO: 425 - DNA, 20 bp, synthetic construct
  source 1..20: mol_type = other DNA
ttaaaaaaaaaaaaaaaaaa

SEQ ID NO: 426 - DNA, 20 bp, synthetic construct
  source 1..20: mol_type = other DNA
ttaatttttttttttttttt

SEQ ID NO: 427 - DNA, 20 bp, synthetic construct
  source 1..20: mol_type = other DNA
ttactttttttttttttttt

SEQ ID NO: 428 - DNA, 20 bp, synthetic construct
  source 1..20: mol_type = other DNA
ttatatatatatatatatat

SEQ ID NO: 429 - DNA, 20 bp, synthetic construct
  source 1..20: mol_type = other DNA
ttattattattattattttt

SEQ ID NO: 430 - DNA, 20 bp, synthetic construct
  source 1..20: mol_type = other DNA
ttatttatttatttattttt

SEQ ID NO: 431 - DNA, 20 bp, synthetic construct
  source 1..20: mol_type = other DNA
ttgctttttttttttttttt

SEQ ID NO: 432 - DNA, 20 bp, synthetic construct
  source 1..20: mol_type = other DNA
ttgtgtgtgtgtgtgtgtgt

SEQ ID NO: 433 - DNA, 20 bp, synthetic construct
  source 1..20: mol_type = other DNA
ttgtttgtttgtttgttttt

SEQ ID NO: 434 - DNA, 20 bp, synthetic construct
  source 1..20: mol_type = other DNA
ttgttttgttttgttttttt

SEQ ID NO: 435 - DNA, 20 bp, synthetic construct
  source 1..20: mol_type = other DNA
tttaaaaaaaaaaaaaaaaa

SEQ ID NO: 436 - DNA, 20 bp, synthetic construct
  source 1..20: mol_type = other DNA
tttatatatatatatatata

SEQ ID NO: 437 - DNA, 20 bp, synthetic construct
  source 1..20: mol_type = other DNA
tttattattattattattat

SEQ ID NO: 438 - DNA, 20 bp, synthetic construct
  source 1..20: mol_type = other DNA
tttatttatttatttatttt

SEQ ID NO: 439 - DNA, 20 bp, synthetic construct
  source 1..20: mol_type = other DNA
tttattttattttatttttt

SEQ ID NO: 440 - DNA, 20 bp, synthetic construct
  source 1..20: mol_type = other DNA
tttgtgtgtgtgtgtgtgtg

SEQ ID NO: 441 - DNA, 20 bp, synthetic construct
  source 1..20: mol_type = other DNA
tttgttgttgttgttgttgt

SEQ ID NO: 442 - DNA, 20 bp, synthetic construct
  source 1..20: mol_type = other DNA
tttgtttgtttgtttgtttt

SEQ ID NO: 443 - DNA, 20 bp, synthetic construct
  source 1..20: mol_type = other DNA
tttgttttgttttgtttttt

SEQ ID NO: 444 - DNA, 20 bp, synthetic construct
  source 1..20: mol_type = other DNA
tttgtttttgtttttgtttt

SEQ ID NO: 445 - DNA, 20 bp, synthetic construct
  source 1..20: mol_type = other DNA
ttttatttatttattttttt

SEQ ID NO: 446 - DNA, 20 bp, synthetic construct
  source 1..20: mol_type = other DNA
ttttattttattttattttt

SEQ ID NO: 447 - DNA, 20 bp, synthetic construct
  source 1..20: mol_type = other DNA
ttttgttgttgttgttgttg

SEQ ID NO: 448 - DNA, 20 bp, synthetic construct
  source 1..20: mol_type = other DNA
ttttgtttgtttgtttgttt

SEQ ID NO: 449 - DNA, 20 bp, synthetic construct
  source 1..20: mol_type = other DNA
ttttgttttgttttgttttt

SEQ ID NO: 450 - DNA, 20 bp, synthetic construct
  source 1..20: mol_type = other DNA
ttttgttttttttttttttt

SEQ ID NO: 451 - DNA, 20 bp, synthetic construct
  source 1..20: mol_type = other DNA
tttttgttgttgttgttgtt

SEQ ID NO: 452 - DNA, 20 bp, synthetic construct
  source 1..20: mol_type = other DNA
tttttgtttgtttgtttgtt

SEQ ID NO: 453 - DNA, 20 bp, synthetic construct
  source 1..20: mol_type = other DNA
tttttgtttgtttgtttttt

SEQ ID NO: 454 - DNA, 20 bp, synthetic construct
  source 1..20: mol_type = other DNA
tttttgttttgttttgtttt

SEQ ID NO: 455 - DNA, 20 bp, synthetic construct
  source 1..20: mol_type = other DNA
tttttgtttttttttttttt

SEQ ID NO: 456 - DNA, 20 bp, synthetic construct
  source 1..20: mol_type = other DNA
ttttttgtttgtttgtttgt

SEQ ID NO: 457 - DNA, 20 bp, synthetic construct
  source 1..20: mol_type = other DNA
ttttttgtttgtttgttttt

SEQ ID NO: 458 - DNA, 20 bp, synthetic construct
  source 1..20: mol_type = other DNA
ttttttgttttgttttgttt

SEQ ID NO: 459 - DNA, 20 bp, synthetic construct
  source 1..20: mol_type = other DNA
ttttttgttttttttttttt

SEQ ID NO: 460 - DNA, 20 bp, synthetic construct
  source 1..20: mol_type = other DNA
tttttttgtttgtttgtttg

SEQ ID NO: 461 - DNA, 20 bp, synthetic construct
  source 1..20: mol_type = other DNA
tttttttgtttgtttgtttt

SEQ ID NO: 462 - DNA, 20 bp, synthetic construct
  source 1..20: mol_type = other DNA
tttttttgttttgttttgtt

SEQ ID NO: 463 - DNA, 20 bp, synthetic construct
  source 1..20: mol_type = other DNA
tttttttgtttttttttttt

SEQ ID NO: 464 - DNA, 20 bp, synthetic construct
  source 1..20: mol_type = other DNA
ttttttttgtttgtttgttt

SEQ ID NO: 465 - DNA, 20 bp, synthetic construct
  source 1..20: mol_type = other DNA
ttttttttttttcttttttt

SEQ ID NO: 466 - DNA, 20 bp, synthetic construct
  source 1..20: mol_type = other DNA
tttttttttttttctttttt

SEQ ID NO: 467 - DNA, 20 bp, synthetic construct
  source 1..20: mol_type = other DNA
ttttttttttttttcttttt

SEQ ID NO: 468 - DNA, 20 bp, synthetic construct
  source 1..20: mol_type = other DNA
tttttttttttttttttaaa

SEQ ID NO: 469 - DNA, 20 bp, synthetic construct
  source 1..20: mol_type = other DNA
tttttttttttttttttaat

SEQ ID NO: 470 - DNA, 20 bp, synthetic construct
  source 1..20: mol_type = other DNA
tttttttttttttttttgct

SEQ ID NO: 471 - DNA, 20 bp, synthetic construct
  source 1..20: mol_type = other DNA
tttttttttttttttttggt

SEQ ID NO: 472 - DNA, 20 bp, synthetic construct
  source 1..20: mol_type = other DNA
ttttttttttttttttttaa

SEQ ID NO: 473 - DNA, 20 bp, synthetic construct
  source 1..20: mol_type = other DNA
ttttttttttttttttttac

SEQ ID NO: 474 - DNA, 20 bp, synthetic construct
  source 1..20: mol_type = other DNA
ttttttttttttttttttcc

SEQ ID NO: 475 - DNA, 20 bp, synthetic construct
  source 1..20: mol_type = other DNA
ttttttttttttttttttct

SEQ ID NO: 476 - RNA, 20 nt, synthetic construct
  source 1..20: mol_type = other RNA
ttttttttttttttttttgc

SEQ ID NO: 477 - DNA, 20 bp, synthetic construct
  source 1..20: mol_type = other DNA
ttttttttttttttttttgg

SEQ ID NO: 478 - DNA, 20 bp, synthetic construct
  source 1..20: mol_type = other DNA
ttttttttttttttttttgt

SEQ ID NO: 479 - DNA, 20 bp, synthetic construct
  source 1..20: mol_type = other DNA
ttttttttttttttttttta

SEQ ID NO: 480 - DNA, 20 bp, synthetic construct
  source 1..20: mol_type = other DNA
tttttttttttttttttttc